(19) United States
(12) Patent Application Publication
(10) Pub. No.: US 2006/0258606 A1
(43) Pub. Date: Nov. 16, 2006

(54) Transforming growth factor-beta response element decoys and methods

(76) Inventors: Kenneth R. Cutroneo, Burlington, VT (US); Jen-Fu Chiu, Hong Kong (CN)

Correspondence Address: DICKSTEIN SHAPIRO LLP, 1825 EYE STREET NW, Washington, DC, 20006-5403, US

(21) Appl. No.: 11/288,429

(22) Filed: Nov. 29, 2005

Related U.S. Application Data
(62) Division of application No. 10/845,442, filed on May 14, 2004, now abandoned.
(60) Provisional application No. 60/470,474, filed on May 14, 2003.

(51) Int. Cl. A61K 48/00; C07H 21/04

(52) U.S. Cl. 514/44; 514/81; 536/23.5; 435/325

(57) ABSTRACT

Products and methods for treating fibrosis, liver cirrhosis and cancer, and for altering or modulating the expression of genes regulated by transforming growth factor—beta (TGF-β) are provided. Nucleic acid molecules comprising TGF-response elements have a high affinity for transcription factors modulated by TGF-β. In one embodiment, nucleic acid molecules comprising TGF-β response elements can be used as decoys to alter the expression of genes regulated by TGF-β (e.g., proα1(I) collagen) and treat a variety of conditions related to aberrant expression of TGF-β regulated genes (e.g., fibrosis, liver cirrhosis, tumors, cancers).

### CROSS-REFERENCE TO RELATED APPLICATIONS

This application claims priority to Provisional Application No. 60/470,474, filed on May 14, 2003, which is hereby incorporated by reference in its entirety.

### FIELD OF THE INVENTION

In an embodiment of the invention, molecules and related methods are provided that can be used as decoys for the transforming growth factor-β (TGF-β) response element. In one embodiment, the decoy TGF-β response element can bind a target transcription factor and alters expression of genes that are regulated by TGF-β. Specifically, the present invention provides nucleic acid molecules that have high affinity for transcription factors, such as activators and repressors, that are modulated by TGF-β. In particular, the present invention provides a nucleic acid molecule comprising one or more TGF-β response elements. The molecules of the present invention may be introduced into cells as decoy cis-elements to bind transcription factors with high affinity and alter expression of genes that are regulated by TGF-β. Accordingly, the present invention relates to cells comprising a nucleic acid molecule comprising one or more TGF-β response elements. In a preferred embodiment, the present invention relates to methods of regulating expression of proα1(I) collagen. The present invention further relates to pharmaceutical compositions and therapeutic agents comprising molecules that bind to transcription factors that are modulated by TGF-β. The present invention also provides methods for preventing, treating, managing, or ameliorating diseases and conditions associated with aberrant expression of TGF-β regulated genes, e.g., proα1(I) collagen, including, but not limited to, tissue fibrosis, frank fibrosis, liver cirrhosis, tumors and/or cancers of all types, including but not limited to, hepatocellular carcinoma. Moreover, the methods of the present invention comprise administering the therapeutic agent of the present invention alone, or in combination with surgery, or in further combination with standard and experimental therapies, including but not limited to, chemotherapies, hormonal therapies, biological therapies/immunotherapies, radiation therapies, embolization and/or chemoembolization therapies.

### BACKGROUND OF THE INVENTION

Fibrosis and Liver Cirrhosis. In response to tissue injury, there are four phases for tissue repair and regeneration; the clotting phase, the inflammatory phase, the proliferative phase and the remodeling-crosslinking phase. The inflammatory phase is mediated by 2-Cyclooxgenase (Cox-2) which is overexpressed in a vast number and tumor types. The inflammatory phase leads to the proliferative phase in which fibroblasts and other reparative cells migrate into the injured area, proliferate and synthesize the components of the extracellular matrix including collagen. Excessive synthesis of extracellular matrix components can result in tissue fibrosis.

Tissue fibrosis is guarded against by the proper regulatory controls remaining in place during wound repair and regeneration. The growth factors, such as transforming growth factor-β (TGF-), insulin-like growth factor (IGF-1), platelet-derived growth factor (PDGF), and epidermal growth factor (EGF) are of paramount importance during the normal wound healing process in skin and internal organs when neutrophils and macrophages accumulate at the wound site due to the presence of these chemotactic and mitogenic factors. Macrophages secrete TGF-β, basic fibroblast growth factor (bFGF) and vascular endothelial growth factor (VEGF), so as to allow fibroblast and endothelial cell proliferation. Fibroblast and other reparative cells synthesize, accumulate and secrete the components of the extracellular matrix. If the levels of the growth factors remain in check, normal tissue repair follows. This process is normally followed by the production of proteases, decreased synthesis of protease inhibitors, tissue maturation, remodeling and reorganization.

Abnormal regeneration of the epithelium may result from failure to replicate the components of the extracellular matrix. Therefore, provisional repair may be followed by excessive collagen synthesis, resulting in tissue fibrosis and scarring. This irreversible fibrosis results from an over-expression of growth factors, predominantly TGF-β, which stimulates fibroblasts to proliferate and to synthesize the components of the extracellular matrix, resulting in loss of tissue function of internal organs, such as liver and lung. Scarring of skin is a cosmetic problem.

Among the conditions resulting from fibrosis is hepatocellular carcinoma (HCC) which occurs in frequent association with liver fibrosis since this is one of the most important factors in onset and progression of hepatic carcinogenesis. Hepatocellular carcinoma (HCC) is one of the ten most common cancers in the world with a pronounced geographic variation. One of the most prevalent causes in the development of HCC in fibrotic patients is hepatitis resulting from viral infection (Alberti et al., 1995, J. Hepatology 22:38-41; Alter et al., 2000, Sem. Liver Dis. 20:17-35). The onset of HCC results primarily from hepatitis type C and B viral infection which is less prevalent in Western countries with a higher prevalence in South Africa, Asia and the Pacific Islands (Keehn et al., 1991, Cancer Nursing 14:163-174). HCC is the leading cancer in men in Taiwan (Kohno, H. et al., 2002, Expert Opin. Therap. Target 6:483-490). However, type C viral infection is becoming an important risk factor for hepatitis in the United States.

The incidence of HCC in North America is increasing as a result of fibrosis in patients with hepatitis C virus infection (Baffis et al., 1999, Ann. Intern. Med. 131:696-701). Although there is an additive and independent effect of hepatitis C and B viral infection on HCC development (Tsai et al., 1997, Brit. J. Cancer 76:968-974), different incidence profile, risk factors and patterns of morphogenesis of HCC development in hepatitis B and C viral associated cirrhosis suggests different mechanisms of carcinogenesis (Benvegriu et al., 2001, Antiviral Res. 52:199-207; Ikeda et al., 1998, J. Hepatology 28:930-938).

Hepatitis infection results in liver cirrhosis, a diffuse fibrosis which is characterized by an architectural distortion, the development of nodules which range from benign regenerative nodules to premalignant dysplastic nodules to overtly malignant HCC. Understanding the natural history of HCC has come from prospective studies with progressive cirrhosis, a diffuse process of liver fibrosis. The course of HCC may vary in patients and in different geographical areas (Colombo et al., 1992, Ital. J. Gastroenterol. 24:95-99). Since patients suffering from liver cirrhosis and frank fibrosis are at high risk for the development of HCC, effective treatment regiments become ever so more important.

Two major factors noted in causing the pathogenesis of HCC are chronic hepatitis and hepatic fibrosis. In patients with viral hepatitis, prognosis is worsened in conjunction with schistosomiasis (Hammad et al., 1990, J. Trop. Ped. 36:126-127). The association of virally-induced hepatitis, liver fibrosis induced by schistosomiasis and the development of HCC has been demonstrated (Mabrouk, 1997, Dis. Mark. 13:177-182; Badawi et al., 1999, Anticancer Res. 19:4565-4569), with viral hepatitis and schistosomiasis-induced granulomatous liver inflammation being primary risk factors for HCC.

Schistosomiasis affects hundreds of millions of people in the tropics. Eggs trapped in the portal presinusoidal venules induce a granulomatous inflammatory reaction with subsequent fibrosis. Although early manifestations of the disease such as hepatomegaly resolve relatively quickly, in the later forms of the disease such as liver fibrosis resolution resolves very slowly or not at all (Boros, 1989, Clin. Microbiol. Rev. 2:250-269; Weinstock, 1992, Immunol. Invest. 21:455-475). Therefore it becomes ever so important during the later form of the disease to treat the fibrogenic process.

TGF-β and collagen expression. TGF-β is a growth and differentiation factor that acts both as a tumor suppressor and a contributor to tumor invasion and metastasis (Akhurst, 2002, J. Clin. Invest. 109:1533-1536). TGF-β also participates in wound healing by stimulating fibroblasts and other cells to synthesize and secrete extracellular matrix factors, including collagen. TGF-β reacts with receptors on the cell membrane resulting in signals to the nucleus causing transcription of specific genes, including cyclooxygenase, lysyl oxidase, proα1(I) collagen gene, proα2(I) collagen, elastin and connective tissue growth factor (CTGF)(Yang et al., 1997, Biochim. Biophys. Acta 1350:287-292; Grotendorst et al., 1996, Cell Growth and Differ. 7:469-480). The proα1(I) collagen gene comprises a TGF-β response element in the 5′ flanking region. The sequence of events for wound healing involves the TGF-β activator protein complex as a trans-activating factor, binding to the TGF-β response element, thereby inducing the transcription of the proα1(I) collagen gene. TGF-β may also regulate transcription of collagen genes indirectly by aiding in the binding of other transcription factors to regulatory elements in collagen genes (Lindahl et al., 2002, J. Biol. Chem. 277:6153-6161).

The therapeutic basis for treatment of any disease derives from an understanding of the cellular and molecular biology of the disease state. HCC develops from hepatocytes. The stroma of the cancerous lesion is infiltrated with proliferating myofibroblasts (Terada et al., 1996, J. Hepatology 24:706-712; Bald et al., 1998, Cell Mol. Biol. 44:627-633) which produce extracellular proteins that lead to liver fibrosis. The main source of myofibroblasts is the perisinusoidal stellate cell which responds to injury with a pleiotypic change termed activation. Hepatic stellate cell recruitment and activation is regulated by the tumoral hepatocyte cells (Faouzi et al., 1999, Lab Invest. 79:485-493). Activation is accomplished by cytokines and the extracellular matrix itself TGF-β is the predominant cytokine in this process. Also of importance is a “fetal” isoform of fibronectin which arises from sinusoidal endothelial cells. It is stimulated by TGF-β and acts directly on stellate cells to promote their activation (Bissell, 2001, Exp. Mol. Med. 33:179-190). In addition, HCC has reduced expression of the type II TGF-β receptor which may provide a selective growth advantage to HCC to escape the inhibitory growth signals of TGF-β (Ueno et al., 2001, Intl. J. Oncol. 18:49-55). Lamellar fibrosis in the fibrolamellar variant of HCC may be due to the action of TGF-β produced by the tumor cells (Orsatti et al., 1997, Liver 17:152-156).

The importance of altered collagen parameters and other metabolic processes in HCC and liver fibrosis has been reported. Collagen glucosyltransferase is increased in primary liver carcinoma and in murine schistosomiasis-induced liver fibrosis (Bolarin, 1991, Acta Physiol. Hung. 77:113-120). While this intracellular enzyme involved in collagen synthesis is increased, the serum level of the tissue inhibitor of metalloproteinases, the primary regulator of mammalian collagenase activity, is increased in chronic liver disease and in HCC (Murawaki et al., 1993, Clin. Chim. Acta 218:47-58). The N-terminal propeptide of type III procollagen, an indicator of collagen synthesis, is elevated in the serum of patients with schistosomiasis infected livers (Shahin et al., 1992, Hepatology 15:637-644). In addition, a liver connective tissue cell line derived from schistosomal granulomas was shown to produce proteoglycans (Silva et al., 1992, Biochim Biophys Acta 1138:133-142). Serum collagenase activity decreased as active liver fibrosis occurred since the tissue inhibitor of metalloproteinase increased (Murawaki et al., 1993, J. Hepatology 18:328-334). Tenacin, an oligomeric glycoprotein of the extracellular matrix, is increased in HCC (Yamada et al., 1992, Liver 12:10-16) and in serum in chronic liver disease (Yamauchi et al., 1994, Liver 14:148-153). The expression of cellular fibronectin is increased in HCC (Matsui et al., 1997, Hepatology 27:843-853). Serum type IV collagen is elevated in primary and metastatic liver cancer and in liver cirrhosis (Hong et al., 1995, Anticancer Res. 15:2777-2780). Plasma TGF-β is elevated in HCC and plays an important role in the altered collagen metabolism in HCC (Murawaki et al., 1996, Gastroenterol Hepatol 11:443-450).

The Chinese herbal medicine Sho-saiko-to is mixture of seven herbal preparations which is used in Japan for chronic hepatitis and cirrhosis (Shimizu, I., 2000, J. Gastroenterol. Hepatol. 15 suppl: D84-9). It protects against hepatic fibrosis and HCC. Halofuginone, a type I collagen inhibitor improves the survival of patients with HCC and cirrhosis undergoing surgical resection (Spira, G., Mawasi, N., Paizi, M., Anbinder, N., Genina, O., Alexiev, R. and Pines, M., 2002, J. Hepatology 37:331). To date glucocorticoids are the only modern therapeutic agents for the treatment of fibrotic diseases. However, these drugs possess numerous adverse side effects.

Methods Of Gene Regulation. A major focus of cellular and molecular research has concentrated on developing means to regulate gene expression (i.e., gene transcription and translation) in an effort to prevent, treat, manage, ameliorate, and cure a variety of diseases and conditions associated with aberrant gene expression. The goal is that the up- or down-regulation of specific genes will alter or circumvent the molecular mechanisms underlying these diseases and conditions. Currently, several methods have been developed to regulate and control gene expression at either the transcriptional or translational steps. Each of these methods suffers from significant drawbacks.

One means of regulating gene expression is to use chemicals that alter the expression of all genes within a cell, tissue, or organism. For example, cycloheximide blocks the peptidyl transferase reaction on eukaryotic ribosomes and acts as a general inhibitor of translation (i.e., the translation of all genes within treated cells is inhibited). Likewise, α-amantin globally blocks mRNA synthesis by binding to eukaryotic RNA polymerase II. Furthermore, actinomycin D is capable of blocking RNA synthesis by intercalating into guanine-cytosine base pairs and disrupting transcription; netropsin and distamycin A block transcription by binding to DNA and blocking RNA polymerase; and acridines, such as proflavine, inhibit RNA synthesis by blocking the formation of the DNA/RNA polymerase complex. Because these chemicals prevent the expression of all genes, any prolonged treatment results in the loss of critical factors needed to maintain the cells, leading to irreparable damage or cell death. To overcome these drawbacks, methods of regulating the expression of specific genes or gene families must be developed.

Another means of regulating gene expression is to activate or repress the signal transduction pathways that are responsible for regulating gene transcription. By activating or inhibiting important steps in the pathways (e.g., binding of signaling molecules to receptors, entry of signaling molecules into cells or nuclei, covalent modification of enzymes, or release or sequestration of ions from organelles), gene expression can be activated or repressed.

The displacement of a modulator protein from transcriptional regulatory sites provides a strategy for gene-specific activation or repression. For example, procaryotic repressors can function as negative regulators of eukaryotic promoters (Brown et al., 1987, Cell 49:603 612; Hu et al., 1988, Gene 62:301 313). Trans-dominant mutants, which retain the ability to bind to cis-regulatory DNA sequences (e.g., enhancers) but lack functional transcriptional activation domains, can also be designed to regulate gene expression through such displacement. These mutant transcription factors compete with their functional, wild-type counterparts for binding to the enhancer sequences, and thereby modulate the activation or repression of the target gene.

Thus, the art remains in need of means for regulating target expression of genes regulated by TGF-β to control and treat human fibrotic diseases, such as liver cirrhosis and hepatocellular cancer.

### BRIEF SUMMARY OF THE INVENTION

Embodiments of the present invention are based, in part, on the observations by the present inventors that expression of proα1(I) collagen is inhibited by administration of TGF-β response element decoy (e.g., a double stranded nucleic acid molecule comprising one or more TGF-β response elements having the nucleic acid sequence of 5′-TGCCCACGGCCAG-3′, SEQ ID NO: 1) to an animal, whereby the TGF-β response element decoy competes with the TGF-β response element in the 5′ flanking region of endogenous proα1(I) collagen for binding to transcription factors that are modulated by TGF-β. Consequently, the expression of proα1(I) collagen and other specific genes that are involved in liver cirrhosis are suppressed. Accordingly, embodiments of the invention relate to preventing, treating, managing or ameliorating liver cirrhosis in an animal by regulating the formation of fibrosis in liver cells. A preferred embodiment comprises nucleic acid molecules and methods for reducing the symptom of liver cirrhosis by inhibiting expression of proα1(I) collagen regulated by transforming growth factor-β (TGF-β) in liver cells.

Embodiments of the present invention provide molecules comprising one or more TGF-β response elements that can be used as decoy TGF-β response elements to bind transcription factors that are modulated by TGF-β. In specific embodiments, the present invention provides nucleic acid molecules, e.g., single stranded or double stranded nucleic acid molecule or polynucleotides, with high affinity for transcription factors that are modulated by TGF-β. In one embodiment, the present invention provides a nucleic acid molecule that competes with endogenous TGF-β response element for binding to one or more transcription factors. In some embodiments, the nucleic acid molecules comprise DNA, although all nucleic acid molecules (e.g., RNA) are contemplated by the presently claimed invention.

In a preferred embodiment, the TGF-β response element decoy comprises a double stranded nucleic acid molecule, i.e., two oligonucleotides that are capable of hybridizing to each other to form a duplex. The double stranded nucleic acid molecule comprises one or more TGF-β response element sequences. In particularly preferred embodiments, the double stranded nucleic acid molecules comprise 5′-TGCCCACGGCCAG-3′ (SEQ ID NO. 1) and its complementary sequence. However, all double-stranded nucleic acid molecules that can compete with a TGF-β response element for binding to one or more transcription factors are contemplated by the presently claimed invention. In some embodiments, the nucleic acid molecules comprise one or more single stranded oligonucleotides. In preferred embodiments, the single stranded oligonucleotides comprise one or more TGF-β response element sequences. In particularly preferred embodiments, the single stranded oligonucleotides comprise 5′-TGCCCACGGCCAG-3′ (SEQ ID NO. 1). However, all single stranded olignucleotides that can compete with a TGF-β response element for binding to one of more transcription factors are contemplated by the presently claimed invention.

In other embodiments, the TGF-β response element decoy comprises one or more hairpin-forming single-stranded oligonucleotides. In other embodiments, the TGF-β response element decoy comprises two hairpin-forming oligonucleotides complementary to one another in a manner wherein combining the two hairpin-forming oligonucleotides produces a cruciform structure.

Although not required by the presently claimed invention, in some embodiments the nucleic acid molecules contain modified phosphodiester bonds. In some embodiments, these modified phosphodiester bonds are selected from the group consisting of phosphorothioate, phosphoramidate, and methyl phosphate derivatives.

The present invention further relates to pharmaceutical compositions and therapeutic agents comprising TGF-β response element decoys. The present invention further relates to a cell comprising a TGF-β response element decoy.

The presently claimed nucleic acid molecules may be introduced into a cell as decoy TGF-β response elements to bind transcriptions factors modulated by TGF-β and alter expression of genes. Accordingly, the present invention preferably provides a method for altering expression of genes that are regulated by TGF-β in a cell, said method comprising administering to the cell one or more TGF-β response element decoys. In certain embodiments of the presently claimed method, the cell is a cancer cell. In some embodiments, the nucleic acid molecule of the present invention is introduced into the cell via injection, direct exposure, or transfection.

The presently claimed invention further provides methods for preventing, treating, managing, or ameliorating diseases and conditions associated with aberrant expression of a gene regulated by TGF-β including, but not limited to, tissue fibrosis, frank fibrosis, liver cirrhosis, tumors and/or cancers of all types, including, but not limited to, hepatocellular cancer, in a patient in need thereof, said method comprising administering to said patient a therapeutically effective amount of an isolated nucleic acid molecule comprising an oligonucleotide or oligomer comprising a sequence which comprises a TGF-β response element, i.e., a TGF-β response element decoy.

Definitions. As used herein, the term “nucleic acid” refers to any nucleic acid containing molecule including, but not limited to DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, and 2,6-diaminopurine.

As used herein, the term “oligonucleotide”, refers to a short polymer or chain of chemically linked nucleotides. Oligonucleotides are typically less than 100 residues long (e.g., between 15 and 50), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can be of secondary, tertiary and quaternary form, including, but are not limited to, duplexes, hairpins, cruciforms, bends, and triplexes. When present in double stranded form, it is understood that the two complementary strands are present, i.e., a “double-stranded nucleic acid molecule” refers to two complementary oligonucleotides which form a duplex.

As used herein, the term “oligomer”, refers to a molecule comprising a plurality of units derived from molecules of lower relative molecular mass. This term encompasses both oligonucleotides and chains of non-nucleotide units.

As used herein, the term “transcription factor” or “transactivating factor” refers to proteins that interact with one another and RNA polymerase enzyme to modulate transcription. Transcription factors target genes by recognizing specific DNA regulatory sequences (e.g., response elements) or other transcription factors. Transcription factors are often referred to as “trans-factors” that interact with “cis-elements” (e.g., response elements) because they are typically produced from genes located distantly (trans) from their sites of regulation (cis). Transcription factors that are involved in cancer are discussed in Nebert, 2002, Toxicology, 181-182:131-141; Darnell, Jr., 2002, Nature Rev. 2:740-749. Representative transcription factors include AP1, ATF, E2F, ETS, FOS, NFκB, SP1 and STAT family members. It is recognized that transcription factors can act as activators, i.e. stimulate transcription or repressors, i.e. inhibit transcription. Thus, decoy response elements which compete for binding for activators will inhibit transcription, while decoy response elements which compete for binding for repressors will stimulate transcription.

As used herein, a “TGF-β transactivating factor” is a transactivating factor modulated or induced by TGF-β signaling, i.e., the binding of TGF-β to its receptor and subsequent downstream events.

As used herein, the term “TGF-β response element” refers to a nucleotide sequence which binds transcription factors, specifically TGF-β transactivating factors, i.e., transcription factors modulated by TGF-β. These sequences include, but are not limited to, non-native sequences, consensus sequences (e.g., 5′ TGCCCACGGCCAG-3′ (SEQ ID NO. 1)), as well as TGF-β response elements that are associated with native genes. Moreover, these sequences encompass variants of TGF-β response element sequences, resulting from e.g., base substitution(s), addition(s) or deletion(s), and derivatives or analogues thereof, and sequences which hybridize to the consensus sequence or native TGF-β response elements under high, medium, or low stringency.

As used herein, the terms “response element decoy” and “TGF-β response element decoy” refer to molecules that bind to or interact with transcription factors that are modulated by TGF-β and prevent their binding to endogenous regulatory sequences, e.g., TGF-β response element sequences. Decoys include nucleic acid sequences, including, but not limited to, nucleic acid molecules that comprise TGF-β response element sequences. Such nucleic acid molecules include, but are not limited to, single stranded or double stranded nucleic acid molecules comprising one or more TGF-β response element sequence, single stranded oligonucleotides that form hairpin structures such that a duplex binding site for the transcription factor is generated, and one or more oligonucleotides that form a cruciform structure such that one or more binding sites for the transcription factor are generated.

As used herein, the term “duplex”, in reference to oligonucleotides, refers to regions that are double stranded through hybridization of complementary base pairs. The term “hairpin” refers to double-stranded nucleic acid structures formed by base-pairing between regions of the same strand of a nucleic acid molecule. The regions are arranged inversely and can be adjacent or separated by noncomplementary sequence (i.e., thus forming a loop structure or “stem-loop”). The term “cruciform” refers to structures formed in double-stranded nucleic acids by inverted repeats separated by a short sequence. Cruciform structures can be generated through the hybridization of two or more hairpin structures where the hairpin duplex and loop comprise the short sequence separating the inverted repeats. Cruciform structures can comprise one or more nucleic acid molecules.

As used herein, the term “high affinity” refers to the non-random interaction of a molecule with itself or another molecule. Molecules with affinity for one another will tend to “bind” (i.e., chemically associate through weak or strong chemical interactions) and form a stable complex. For example, a transcription factor will have high affinity for polynucleotide sequences that correspond to its DNA binding domain and low affinity for other nucleic acid sequences.

As used herein, the term “derivative” refers to any pharmaceutically acceptable homolog, analogue, or fragment corresponding to the composition of the invention.

As used herein, the term “cancer” describes a disease state in which a carcinogenic agent or agents causes the transformation of a healthy cell into an abnormal cell, which is followed by an invasion of adjacent tissues by these abnormal cells, and which may be followed by lymphatic, cerebral spinal fluid, or blood-borne spread of these abnormal cells to regional lymph nodes and/or distant sites, i.e., metastasis.

As used herein, the term “tumor” or “growth” means increased tissue mass, which includes greater cell numbers as a result of faster cell division and/or slower rates of cell death. Tumors may be malignant or non-malignant cancers.

As used herein, the phrases “treating cancer” and “treatment of cancer” mean to inhibit the replication of cancer cells, inhibit the spread of cancer, decrease tumor size, lessen or reduce the number of cancerous cells in the body, or ameliorate or alleviate the symptoms of the disease caused by the cancer. The treatment is considered therapeutic if there is a decrease in mortality and/or morbidity, or a decrease in disease burden manifest by reduced numbers of malignant cells in the body.

As used herein, the phrases “preventing cancer” and “prevention of cancer” mean to prevent the occurrence or recurrence of the disease state of cancer. As such, a treatment that impedes, inhibits, or interferes with metastasis, tumor growth, or cancer proliferation has preventive activity.

As used herein, the phrase “therapeutics” or “therapeutic agents” refers to any molecules, compounds or treatments that assist in the treatment of a disease. The treatment protocol may include, but is not limited to, radiation therapy, dietary therapy, physical therapy, and psychological therapy.

As used herein, the phrase “chemoagent” or “anti-cancer agent” or “anti-tumor agent” or “cancer therapeutic” refers to any molecule, compound or treatment that assists in the treatment of tumors or cancer.

As used herein, the phrase “low dose” or “reduced dose” refers to a dose that is below the normally administered range, i.e., below the standard dose as suggested by the Physicians' Desk Reference, 54th Edition (2000) or a similar reference. Such a lower or reduced dose may be sufficient to inhibit cell proliferation, or demonstrates ameliorative effects in a human, or demonstrates efficacy with fewer side effects as compared to standard cancer treatments. Normal dose ranges used for particular therapeutic agents and standard cancer treatments employed for specific diseases can be found, for example, in the Physicians' Desk Reference, 54th Edition (2000) or in Cancer: Principles & Practice of Oncology, DeVita, Jr., Hellman, and Rosenberg (eds.) 2nd edition, Philadelphia, Pa.: J.B. Lippincott Co., 1985.

As used herein, the phrase “reduced toxicity” refers to the reduced side effects and toxicities observed in connection with administering the nucleic acid molecules of the present invention and other cancer therapeutics for shorter duration and/or at lower dosages when compared to other treatment protocols and dosage formulations, including the standard treatment protocols and dosage formulations as described in the Physicians' Desk Reference, 54th Edition (2000) or in Cancer: Principles & Practice of Oncology, DeVita, Jr., Hellman, and Rosenberg (eds.) 2nd edition, Philadelphia, Pa.: J.B. Lippincott Co., 1985.

As used herein, the phrase “treatment cycle” or “cycle” refers to a period during which a single therapeutic or sequence of therapeutics is administered. In one embodiment encompassing the use of high doses of TGF-β response element decoys, in combination with a standard dose of a therapeutic agent, the preferred period length of time for one treatment cycle is less than 14 days. The present invention contemplates at least one treatment cycle, generally preferably more than one cycle. In some instances, one treatment cycle may be desired, such as, for example, in the case where a significant therapeutic effect is obtained after one treatment cycle.

As used herein, the phrase “pharmaceutically acceptable carrier” refers to a carrier medium that does not interfere with the effectiveness of the biological activity of the active ingredient. Said carrier medium is essentially chemically inert and nontoxic.

As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, the term “carrier” refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These pharmaceutical compositions can take the form of solutions, suspensions, emulsion, tablets, pills, capsules, powders, sustained release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic polyamines and lipids, including, but not limited to N (1(2,3 dioleyloxy)propyl) N,N,N trimethylammonium chloride (DOTMA) and diolesylphosphotidylethanolamine (DOPE). Liposomes are also suitable carriers for the oligomers of the invention. Such pharmaceutical compositions should contain a therapeutically effective amount of the compound, together with a suitable amount of carrier so as to provide the form for proper administration to the patient. The formulation should suit the mode of administration.

As used herein, the phrase “pharmaceutically acceptable salts” refers to salts prepared from pharmaceutically acceptable, essentially nontoxic, acids and bases, including inorganic and organic acids and bases. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2 ethylamino ethanol, histidine, procaine, etc.

### BRIEF DESCRIPTION OF THE DRAWINGS

FIG. 1: Schematic representation of mechanism of action of response element decoys.

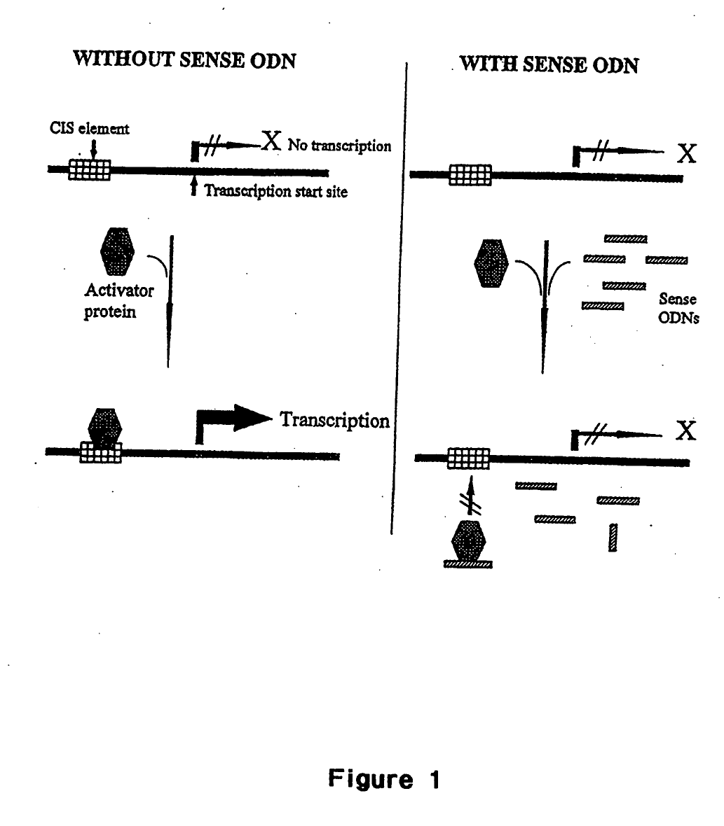

FIG. 2: In vitro competitive binding oligodeoxynucleotide experiment. Fetal rat lung fibroblasts were grown to late log phase and the nuclear extract prepared. The nuclear extract was incubated with 32P double stranded TGF-β oligodeoxynucleotide in the presence of either single-stranded phosphorothioate oligodeoxynucleotides or the double-stranded nucleic acid molecules. The samples were then submitted to gel mobility shift analysis.

FIG. 3: Single stranded response element decoy effect on granuloma weight and granuloma body weight ratio. Rats were implanted with a foam sponge and treated by an intrasponge injection with vehicle or ssPT 24 hours after sponge implantation. The animals received a second injection 24 hours later and euthanized 24 hours thereafter. The granulomatous tissue was weighed and granuloma:body weight ratio determined. The values represent the mean ±SE of data from 4 or 5 animals. *Significantly different from control at p≦0.05.

FIG. 4: Granuloma weight and granuloma:body weight ratio correlate with collagen synthesis. Implanted sponges were injected into the sponge with saline or ssPT for 2 days. Twenty-four after the second injection, the animals received radioactive proline and after 60 minutes the rats were killed. The granulation tissue was assayed for collagen synthesis and the data was analyzed by linear regression analysis. Each point represents the complimentary values for either granuloma weight or granuloma: body weight vs. percent 3H-proline incorporated into collagen for one vehicle treated (◯) or one ssPT treated (●) animal.

FIG. 5: Effect on single-stranded response element decoy on collagen and noncollagen protein synthesis. Animals were treated as described in FIG. 4. The tissue homogenates were digested with collagenase and 3H-proline incorporated into collagen and noncollagen protein was determined. The values represent the mean ±SE of data from 5 to 6 animals. *Significantly different from control at p≦0.05.

FIG. 6: In vivo—antifibrotic intervention. Animals at 6.2 wk of infection received intrahepatically 800 μg in 0.1 ml of dsPT followed by 3 daily iv injections of 200 μg in 0.1 ml. Control animals received buffer. Twenty four hours after the last injection, animals were sacrificed and their livers were examined for the expression of TGF-β, TIMP-1 and collagen I and collagen III messages, as well as total hydroxyproline content.

### DETAILED DESCRIPTION OF THE INVENTION

The present invention provides nucleic acid molecules that bind transcription factors that are modulated by TGF-β. These nucleic acid molecules act as cis-element decoys in that they comprise a sequence that competes for the binding to a transcription factor modulated by TGF-β. In some embodiments, TGF-β response element decoys may comprise more than one TGF-β response element. The invention is further directed to methods of regulating gene expression comprising administering a nucleic acid comprising one or more TGF-β response elements to a cell or other transcriptional system such as in vitro transcription using a nuclear extract. The invention relates to a cell comprising a TGF-β response element. In one embodiment, nucleic acid molecules of the present invention regulate the expression of proα1(I) collagen synthesis. Unregulated collagen synthesis in response to wound healing can lead to tissue fibrosis, which can in turn lead to hepatocellular carcinoma. The present invention also provides methods for preventing or treating a disease or condition associated with tissue fibrosis and/or cancer comprising administering to a subject in need of such treatment a nucleic acid which binds transcription factors modulated by TGF-β. The present invention also provides pharmaceutical compositions and kits for the administration of a TGF-β response element decoy. The invention further provides drug delivery and therapeutic regimens for prophylactic and therapeutic treatments.

TGF-β Response element decoys. TGF-β response element decoys are characterized by the ability to bind transcription factors modulated by TGF-β. When introduced into a cell or other transcription system containing TGF-β and genes regulated by TGFβ, TGF-β response element decoys compete for the binding of transcription factors modulated by TGF-β. By competing for the binding of transcription factors modulated by TGFβ, the transcription of genes regulated by TGF-β is modified. Since binding of transcription factors modulated by TGF-β to promoter regions increases transcription of certain genes, including proα1(I) collagen, TGF-β response element decoys can reduce expression of these genes. A transcription factor does not need to bind directly to a regulatory sequence of a gene, but can aid in the binding of other transcription factors to a regulatory sequence. Response element decoys comprising single stranded nucleic acid molecules that compete with TGF-β response elements for binding to TGF-β have been used to regulate collagen expression (Cutroneo et al., 2000, Wound Rep Reg 8:399-404).

TGF-β response element sequences. The invention contemplates the use of a TGF-β response element decoy comprising one or more TGF-β response elements, its derivatives, analogues or fragments thereof. Preferably, a TGF-β response element decoy is a nucleic acid which binds to transcription factors capable of binding a TGF-β response element, and thereby hinder or prevent binding of the transcription factor to a TGF-β response element. Alternatively, a TGF-β response element decoy can bind transcription factors modulated by TGF-β and prevent TGF-β from mediating the binding of other transcription factors to gene regulatory elements. In one embodiment, the TGF-β response element decoy comprises a TGF-β response element. In a preferred embodiment, the TGF-β response element decoy comprises a rat TGF-β response element consensus sequence, e.g., 5′-TGCCCACGGCCAG-3′ (SEQ ID NO. 1), or a derivative or analogue of a TGF-β response element consensus sequence that retains the ability to compete with native TGF-β response elements for binding to transcription factors. In one embodiment, the TGF-β response element decoy is 5′-AGCCTAACTGCCCACGGCCAGCGACGT-3′ (SEQ ID NO:2), corresponding to positions −1636 to −1610 of the rat proα1(I) collagen promoter. While in some embodiments, the TGF-β response element decoy comprises flanking sequences corresponding to that of the native gene sequence, in other embodiments, flanking sequences can be random sequences or poly(N). In certain embodiments, the TGF-β response element decoy comprises a TGF-β response element that is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides in length. In other embodiments, the TGF-β response element is a human sequence. TGF-β response elements are also found in regulatory sequences from other genes, including, for example, human connective tissue growth factor gene (Eguchi et al., 2002, Biochem. Biophys. Res. Commun. 295:445-451), rat cyclooxygenase-2 (Chen et al., 1999, Life Sci. 64:1231-1242), human cyclooxygenase-2 (Yang et al., 1997, Biochim. Biophys. Acta 1350:287-292) and human TGF-β receptor (Bloom et al., 1996, Biochim. Biophys. Acta, 1312:243-248).

Decoy forms. TGF-β response element decoys suitable for use, and contemplated by the invention, include oligomers which range in size from 16 to 50 bases in length; preferably 17 to 40 bases in length; more preferably from 18 to 38 bases in length; more preferably from 19 to 36 bases in length; and most preferably from 20 to 34 bases in length. In one embodiment, the TGF-β response element decoy is 27 bases in length.

TGF-β response element decoys can be in the form of any nucleic acid or oligomer which can bind to transcription factors modulated by TGF-β. In one embodiment, the response element decoys are in the form of a double-stranded nucleic acid molecule. Double stranded nucleic acid molecules may be in modified form such as dumbbells, described in U.S. Pat. No. 5,683,985, entitled “Oligonucleotide Decoys and Methods Relating Thereto”, herein incorporated by reference in its entirety. Response element decoys can also be photo-crosslinked to provide greater thermal stability as described in Iwase et al., 1997, Nucleic Acids Symp Ser, 37:203-4.

TGF-β response element decoys can also be in single stranded form. In one embodiment, the single stranded response element decoy can be in the form of a single strand which fold back on itself to form a TGF-β response element decoy, i.e., a hairpin structure, described in U.S. Pat. No. 5,683,985, entitled “Oligonucleotide Decoys and Methods Relating Thereto”, herein incorporated by reference in its entirety. In certain embodiments, a single stranded oligonucleotide can bind to transcription factors modulated by TGF-β without forming a double-stranded structure.

Derivatives. Also contemplated by the invention are TGF-β response element sequences that vary from the consensus sequence, by means of base substitutions, deletions and/or additions. Furthermore, TGF-β response element sequences corresponding to identified native TGF-β response elements, and variants thereof (including natural variants), are also encompassed by the present invention. For example, use of native TGF-β response elements, or variants thereof obtained by, for example, base substitutions, deletions or additions, may be useful for targeting specific TGF-β-modulated transcriptional units. Methods for testing derivatives and variants are routine in the art and are discussed below.

In another embodiment, derivatives or variants of TGF-β response elements can be obtained by screening for sequences that hybridize to a TGF-β response element under high stringency, i.e., conditions for hybridization and washing under which nucleotide sequences, which are at least 60% (preferably 65%, 70%, 75%, 80%, 85%, 90%, 95% or greater) identical to each other, typically remain hybridized to each other. Such stringent conditions are known to those skilled in the art, and can be found, for example, in Current Protocols in Molecular Biology, John Wiley & Sons, N.Y. (1989), 6.3.1-6.3.6, which describes aqueous and non-aqueous methods, either of which can be used. Another example of stringent hybridization conditions is hybridization of the nucleotide sequences in 6× sodium chloride/sodium citrate (SSC) at about 45° C., followed by 0.2×SSC, 0.1% SDS at 50-65° C. Particularly preferred stringency conditions are hybridization in 6× sodium chloride/sodium citrate (SSC) at about 45° C, followed by one or more washes in 0.2×SSC, 0.1% SDS at 50° C. Another example of stringent hybridization conditions are hybridization in 6×sodium chloride/sodium citrate (SSC) at about 45° C., followed by one or more washes in 0.2×SSC, 0.1% SDS at 55° C. A further example of stringent hybridization conditions are hybridization in 6×sodium chloride/sodium citrate (SSC) at about 45° C., followed by one or more washes in 0.2×SSC, 0.1% SDS at 60° C. Preferably, stringent hybridization conditions are hybridization in 6×sodium chloride/sodium citrate (SSC) at about 45° C., followed by one or more washes in 0.2×SSC, 0.1% SDS at 65° C. Another preferred example of stringent hybridization condition is 0.5M sodium phosphate, 7% SDS at 65° C., followed by one or more washes at 0.2×SSC, 1% SDS at 65° C.

In another embodiment, the sequence that hybridizes to a TGF-β response element, hybridizes under low stringency conditions, which conditions are known to those skilled in the art (see, e.g., Sambrook et al. Molecular Cloning: A Laboratory Manual 2nd Ed., Cold Spring Harbor Laboratory Press, NY, (1989), 9.47-9.55). For example, low stringency hybridization conditions can be achieved by incubating the nucleotide sequences in 6×sodium chloride/sodium citrate (SSC) at about 45 oC overnight, followed by one or more washes in either 0.2×SSC, 0.1% SDS at room temperature, or 0.2×SSC, 0.1% SDS at 37° C., or 0.2X SSC, 0.1% SDS at 42° C., or 2×SSC, 0.1% SDS at 50° C.

Alternative chemistries. The response element decoys may be RNA or DNA, or derivatives thereof, which may include nucleoside analogues and/or non-nucleoside analogues. The particular form of response element decoy may affect the oligomer's pharmacokinetic parameters such as bioavailability, metabolism, half-life, etc. As such, the invention contemplates nucleic acid molecule derivatives having properties that improve cellular uptake, enhance nuclease resistance, and/or improve binding to the target transactivating factors. Such nucleic acid molecules may possess modifications which comprise, but are not limited to, 2 O′ alkyl or 2-O′ halo sugar modifications, backbone modifications (e.g., methylphosphonate, phosphorothioate, phosphorodithioate, formacetal, 3′ thioformacetal, sulfone, sulfamate, nitroxide backbone, morpholino derivatives and peptide nucleic acid (PNA) derivatives), or derivatives wherein the base moieties have been modified (Egholm et al., 1992, Peptide Nucleic Acids (PNA) Oligomer Analogues With An Achiral Peptide Backbone; Nielsen et al., 1993, “Peptide nucleic acids (PNAs): potential antisense and anti-gone agents”, Anticancer Drug Des 8:53-63). In one embodiment, the nucleic acid molecules of the invention, instead, may be mixed, or chimeric, oligomers. Mixed oligomers may comprise any combination of modified bases. In another embodiment, nucleic acid molecules of the invention comprise conjugates of the oligomers and derivatives thereof (Goodchild, 1990, Bioconjug. Chem. 1(3):165-87).

For in vivo therapeutic use, several types of nucleoside derivatives are available. A phosphorothioate derivative of the nucleic acid molecules of the invention can be useful for in vivo therapeutic use, in part due to the greater resistance to degradation. In one embodiment, the TGF-β response element decoy comprises phosphorothioate bases. In another embodiment, the TGF-β response element decoy contains at least one phosphorothioate linkage. In another embodiment, the TGF-β response element decoy contains at least three phosphorothioate linkages. In a further embodiment, the TGF-β response element decoy contains at least three consecutive phosphorothioate linkages. In yet another embodiment, the TGF-β response element decoy is comprised entirely of phosphorothioate linkages. Methods for preparing nucleic acid derivatives are known in the art. See, e.g., Stein et al., 1988, “Physicochemical properties of phosphorothioate oligodeoxynucleotides”, Nucl. Acids Res., 16:3209 21 (phosphorothioate); Blake et al., 1985, “Inhibition of rabbit globin mRNA translation by sequence specific oligodeoxyribonucleotides”, Biochemistry 24:6132 38 (methylphosphonate); Morvan et al., 1986, “alpha DNA. I. Synthesis, characterization by high field 1H NMR, and base pairing properties of the unnatural hexadeoxyribonucleotide alpha [d(CpCpTpTpCpC)] with its complement beta [d(GpGpApApGpG)]” Nucl. Acids Res. 14:5019 32 (alphadeoxynucleotides); Monia et al., 1993, “Evaluation of 2′ modified oligonucleotides containing 2′ deoxy gaps as antisense inhibitors of gene expression”, J. Biol. Chem. 268:14514 22 (2′ O methyl ribonucleosides); Asseline et al., 1984, “Nucleic acid binding molecules with high affinity and base sequence specificity: intercalating agents covalently linked to oligodeoxynucleotides”, Proc. Natl Acad. Sci. USA 81:3297 3301 (acridine); Knorre et al., 1985, Biochemie 67:783 9; Vlassov et al., 1986, “Nucleic acid binding molecules with high affinity and base sequence specificity: intercalating agents covalently linked to oligodeoxynucleotides”, Nucl. Acids Res. 14:4065 76 (N 2 chiorocethylamine and phenazine); Webb et al., 1986, “Hybridization triggered cross linking of deoxyoligonucleotides”, Nucl. Acids Res. 14:7661 74 (5 methyl N4 N4 ethanocytosine); Boutorin et al., 1984, FEBS Letters 172:43 6 (Fe ethylenediamine tetraacetic acid (EDTA) and analogues); Chi Hong et al., 1986, Proc. Natl. Acad. Sci. USA 83:7147 51 (5 glycylamido 1, 10 o phenanthroline); and Chu et al., 1985, “Nonenzymatic sequence specific cleavage of single stranded DNA”, Proc. Natl. Acad. Sci. USA 82:963 7 (diethylenetriaamine pentaacetic acid (DTPA) derivatives).

The nucleic acid molecules of the invention may contain modified nucleotides including, but not limited to, 5-fluorouracil, 5-bromouracil, 5-chiorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′ methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Additionally, the nucleic acid molecules of the invention may possess modifications which comprise, but are not limited to, hexitol nucleic acids and G-clamp heterocycle modifications. In another embodiment, the nucleic acid molecules comprise conjugates of the nucleic acid molecules and derivatives thereof (Goodchild, 1990, “Conjugates of oligonucleotides and modified oligonucleotides: a review of their synthesis and properties”, Bioconjug. Chem. 1:165-87).

In another embodiment, the nucleic acid molecules of the invention comprise an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625-6641). In yet another embodiment, the nucleic acid molecules of the invention comprise a 2′-O-methylribonucleotide (Inoue et al., 1987, “Synthesis and hybridization studies on two complementary nona(2′ O methyl)ribonucleotides”, Nucleic Acids Res. 15:6131-6148) or a chimeric RNA-DNA analogue (Inoue et al., 1987, “Sequence dependent hydrolysis of RNA using modified oligonucleotide splints and RNase H”, FEBS Lett. 215:327-330).

In certain embodiments, the TGF-β response element decoy can be linked, for example, to peptides (e.g., to target host cell receptors in vivo), or to agents that aid in transport across the cell membrane (See, e.g., Letsinger et al., 1989, “Cholesteryl conjugated oligonucleotides: synthesis, properties, and activity as inhibitors of replication of human immunodeficiency virus in cell culture”, Proc. Natl. Acad. Sci. USA 86:6553-6556; Lemaitre et al., 1987, “Specific antiviral activity of a poly(L lysine) conjugated oligodeoxyribonucleotide sequence complementary to vesicular stomatitis virus N protein mRNA initiation site”, Proc. Natl. Acad. Sci. USA 84:648-652; PCT International Publication No. WO 88/09810) or that aid in transport across the blood-brain barrier (See, e.g., PCT International Publication No. WO 89/10134).

Multimers. In one embodiment, the TGF-β response element decoy comprises two or more TGF-β response element sequences, which may or may not be attached by a linker. In a particular embodiment, the TGF-β response element decoy comprises a doublet or a triplet of a TGF-β response element consensus sequence, e.g., 5′-TGCCCACGGCCAG-3′ (SEQ ID NO. 1), or an analog or derivative thereof. The linker may be a nucleotide or a polynucleotide. In another embodiment, the linker may comprise one or more adenine residues. In another embodiment, the linker may comprise one or more cytosine residues. In another embodiment, the linker may comprise one or more guanine residues. In yet another embodiment, the linker may comprise one or more thymidine residues. In another embodiment, the linker comprises any nucleoside base, including modified nucleosides. In one embodiment, the linker is a nucleotide sequence of at least 1, 2, 3, 4, 5, 6, 7, 8, 9, 10, 11, 12, 13, 14, 15, 16, 17, or 18 bases in length. It is recognized that when multimers are used, the total sequence length of the preferred response element decoys is increased accordingly.

In one embodiment, the linker is absent. In another embodiment, the TGF-β response elements can be attached by means of a linker, which may comprise a non-nucleic acid moiety. These response elements may be attached enzymatically or by crosslinking. Many methods for linking to a nucleic acid are known in the art (See, e.g., Catalog of TriLink BioTechnologies, Inc. 2001). Linkers may be placed at the 3′ end of the nucleic acid molecule, at the 5′ end of the molecule, and/or internally. Linkers useful for producing hybrid oligomers of the present invention include, but are not limited to, quenchers (e.g., TAMRA, DABCYL, QSYTM, DABSYL, DABCYL), amino linkers, thiol linkers, propyl spacers, triethylene glycol spacers, tetraethylene glycol spacers, hexaethylene glycol spacers, and terminal phosphates. Linker lengths can be varied. Moreover, a linker can be further modified, for example, to produce specialized linkers such as the amino linkers, monomethoxytritylaminohexyl phosphoramidite and monomethoxytritylaminododecyl phosphoramidite.

Synthesis. Oligonucleotides can be routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as phosphorothioates. Moreover, oligonucleotides can be routinely ordered through vendors such as Qiagen (Valenica, Calif.) and Integrated DNA Technologies (Coralville, Iowa).

For double stranded nucleic acid molecules, the desired sequence containing a TGF-β response element is synthesized as a single stranded oligonucleotide. Its complement is also synthesized for subsequent annealing.

Once single-stranded oligonucleotides are synthesized, double stranded forms can be made by annealing a single stranded oligonucleotide and its complement. In general, equal molar quantities of these oligonucleotides are suspended in an annealing buffer, heated to remove any secondary structure, then slowly cooled to allow annealing. Oligonucleotides can be heated to their melting temperature or any higher temperature, preferably at least 10° C. higher than the melting temperature. Heating can be from 3-10 minutes or longer. Cooling can be done to room temperature or to 4 over a 30 minute period or greater. In one example, oligonucleotides are suspended in 200 mM NaCl, heated to 95° C. for 7 minutes and then slowly cooled to 4° C. Annealing buffers may contain 10-20 mM Tris-HCl, ph 7.5-8.0, 50-100 mM NaCl or 1 mM MgCl2, and optionally, 1 mM EDTA.

For longer oligonucleotides and oligomers, such as the case for multimers, synthesis can be by polymerase chain reaction from a template containing the multimer sequence, or by restriction enzyme digests of a template containing the multimer sequence. Products can be purified by polyacrylamide gel electrophoresis. Other methods include cloning of nucleic acid molecules in a vector-cell system using standard molecular biology techniques.

Methods of Screening For Compounds That Bind to Transcription Factors Modulated by TGF-β and Modulate Proα1(I) Collagen Expression. The present invention provides sceening assays for potential nucleic acids sequences that bind transcription factors modulated by TGF-β. A vector construct containing the TGF-β response element located in the distal 5′-flanking region of the rat proα1(I) collagen gene can be operably linked to a reporter gene, e.g., a gene encoding chloramphenicol acetyltransferase, β-galactosidase, luciferase or green fluorescent protein. Such constructs can be stably or transiently transfected into cells using methods known in the art. See e.g., Molecular Cloning: a Laboratory Manual, 3rd ed., ed. J. Sambrook. Cold Spring Harbor Laboratory Press, 2000.

After transfection into cells, an response element decoy is added to the media and can be passively taken up by the cells, or the response element decoy can be transiently transfected, e.g., by the calcium phosphate co-precipitation method (see, e.g., Chen et al., 1987, “High-efficiency transformation of mammalian cells by plasmid DNA”, Mol. Cell. Biol. 7:2745-2752). Once inside the cell, the response element decoy competes with the TGF-β response element, thereby inhibiting expression of the reporter gene. Activity of the reporter gene can be assayed by methods routine in the art and by commercially available kits.

Methods for screening can also use native genes containing TGF-β response elements, e.g., collagen genes. Gene expression of proα1(I) collagen can be determined by methods such as real time PCR. Additional screening methods include in vitro transcription assays using nuclear extracts. Collagenase assays are also provided in the art (Newman et al., 1978, “Glucocorticoids selectively decrease the synthesis of hydroxylated collagen peptides”, Mol. Pharmacol. 14:185-198).

Methods for screening can also use radioactively labeled response element decoys. In one embodiment radioactively labeled response element decoys are used in gel shift binding reactions. See Cutroneo et al., 2002, Cancer Lett. 180:145-151.

The compositions of the present invention can be useful as research and diagnostic tools. The response element decoys can be used broadly to study expression of genes regulated by TGF-β For example, many identified genes that have TGF-β response elements 5′ of the coding region, or are otherwise regulated by TGF-β, are amenable to functional studies in which the gene's transcription is influenced by an response element decoy. Similarly, the effects of a response element decoy of the present invention on cells that express the genes regulated by TGF-β can be studied in vitro. Promoter analysis of TGF-β responsive genes to determine sequences important for TGF-β modulation is described in Jin et al., 2000, Methods Mol. Bio. 142:79-95.

In particular, tissue fibrosis and cancer can be studied in vitro using the TGF-β response element decoys. The response element decoys of the invention can be tested on fibrotic or tumor cell lines, for instance, to characterize their sensitivity to TGF-β response element decoy treatment. Similar studies can be performed on non-fibrotic cell lines, correlating the effects of TGF-β response element decoys with the growth characteristics of each cell line. Using the response element decoys of the invention, studies of cell/tissue growth or organization can be performed on tissue cultures or tissue explants as well.

The response element decoys of the present invention also can be used for screening candidate transcription factors or other molecules (e.g., gene regulatory proteins) found in, or associated with, transcriptional complexes. For example, TGF-β response elements corresponding to native sequences, consensus sequences, or variants thereof, can be useful for screening and identifying transcription factors and associated proteins that bind, directly or indirectly, to specific TGF-β response elements. These screening assays can be performed in cell-free or cell-based systems. For example, compounds including a protein array of transcription factors can be screened using the response element decoy of the invention, and the binding detected to identify the compounds that recognize the TGF-β response element. The relative strength of binding can be determined by standard binding assays. The efficacy of transcriptional activation or repression can be determined using constructs comprising the TGF-β response elements and a standard reporter sequence.

The response element decoys of the present invention also can be used for detection assays for transcription factor. Accordingly, one aspect of the present invention relates to detection of the levels of transcription factor in a biological sample (e.g., serum, blood, cells, tissue). Such assays can have prognostic or predictive value to a subject in need of diagnosis, prophylaxis, or treatment. To illustrate, a transcription factor that binds to a particular TGF-β response element can be identified in a biological sample by: obtaining a biological sample from a test subject, contacting the biological sample with an response element decoy of the invention, detecting the binding between the transcription factor and the response element decoy, and isolating the transcription factor.

The TGF-β response element decoys can also be useful for general research purposes. For example, the response element decoys can be labeled and monitored in live cells or tissue by, for example, epifluorescence. Furthermore, in studies of protein-nucleic acid interactions, the response element decoys of the present invention can be used as probes in gel-shift assays.

Methods of Treating or Preventing Disease. The methods and compositions of the present invention can be useful for preventing, inhibiting, or lessening the induction and tissue damage that may be caused by aberrant expression of a gene regulated by TGF-β, such as a collagen gene. The invention provides a method for the use of a TGF-β response element decoy which is administered for the prevention or treatment of a disorder associated with aberrant expression of a gene regulated by TGFβ, including, but not limited to, tissue fibrosis, particularly liver cirrhosis and hepatocellular cancer. Disorders associated with tissue fibrosis encompass diseases including liver cirrhosis, pulmonary fibrosis, sarcoidosis, keloids and renal fibrosis (Border et al., 1994, N Engl J Med 331:1286-92; Branton et al., 1999, Microbes Infect 1:1349-65). Response element decoys may also be effective in treating a broad range of cancers by preventing tumor progression and metastasis.

Cancers and related disorders that can be treated or prevented by methods and compositions of the present invention include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterine); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In one embodiment, TGF-β response element decoys comprise a TGF-β response element, or a variant thereof. In one embodiment, the TGF-β response element decoy comprises a TGF-β response element consensus sequence. In a further embodiment, the TGF-β response element decoy comprises the consensus sequence, 5′-TGCCCACGGCCAG-3′ (SEQ ID NO. 1). In another embodiment, the TGF-β response element decoy comprises multiple copies of the TGF-β response element consensus sequence, which are linked by a nucleotide sequence.

In a specific embodiment, an 27-base phosphorothioate TGF-β response element decoy of the sequence 5′ AGCCTAACTGCCCACGGCCAGCGACGT 3′ (SEQ ID NO:2) is administered.

Dosages The effective dose of TGF-β response element decoy to be administered during a treatment cycle ranges from about 0.01 to 0.1, 0.1 to 1, or 1 to 10 mg/kg/day. Accordingly, the administered dose can be, for example, 0.01, 0.025, 0.05, 0.075, 1, 2, 3, 4, 5, 6, 7, 8, 9 or 10 mg/kg/day. The dose of TGF-β response element decoy to be administered can be dependent on the mode of administration. For example, intravenous administration of a TGF-β response element decoy would likely result in a significantly higher full body dose than a full body dose resulting from a local implant containing a pharmaceutical composition comprising TGF-β response element decoy. In one embodiment, a TGF-β response element decoy is administered subcutaneously at a dose of 0.01 to 10 mg/kg/day; more preferably at a dose of 4 to 9 mg/kg/day; most preferably at a dose of 5 to 7 mg/kg/day. In another embodiment, a TGF-β response element decoy is administered intravenously at a dose of 0.01 to 10 mg/kg/day; more preferably at a dose of 4 to 9 mg/kg/day; most preferably at a dose of 5 to 7 mg/kg/day. In yet another embodiment, a TGF-β response element decoy is administered locally at a dose of 0.01 to 10 mg/kg/day; preferably at a dose of 0.01 to 0.1; more preferably at a dose of 1 to 5 mg/kg/day. It will be evident to one skilled in the art that local administrations can result in lower total body doses. For example, local administration methods such as intratumor administration, intraocular injection, or implantation, can produce locally high concentrations of TGF-β response element decoy, but represent a relatively low dose with respect to total body weight. Thus, in such cases, local administration of a TGF-β response element decoy is contemplated to result in a total body dose of about 0.01 to 5 mg/kg/day.

In another embodiment, a particularly high dose of TGF-β response element decoy, which ranges from about 10 to 20, 20 to 30, or 30 to 50 mg/kg/day, is administered during a treatment cycle. In another embodiment, a particularly high dose of TGF-β response element decoy, which ranges from about 10 to 14, 15 to 20, 21 to 25, 26 to 30, 31 to 35, 36 to 40, 41 to 45 or 46 to 50 mg/kg/day, is administered during a treatment cycle. In a specific embodiment, a TGF-β response element decoy is administered to a subject in need of such treatment at a dose of 10, 11, 12, 13, 14, 15, 16, 17, 18, 19, or 20 mg/kg/day.

Moreover, the effective dose of a particular TGF-β response element decoy may depend on additional factors, including the type of cancer, the disease state or stage of disease, the oligomer's toxicity, the oligomer's rate of uptake by cancer cells, as well as the weight, age, and health of the individual to whom the oligomer is to be administered. Because of the many factors present in vivo that may interfere with the action or biological activity of a TGF-β response element decoy, one of ordinary skill in the art can appreciate that an effective amount of a TGF-β response element decoy may vary for each individual.

In another embodiment, a TGF-β response element decoy is at a dose which results in circulating plasma concentrations of the TGF-β response element decoy which is at least 20, 25, 30, 35, 40, 45, or 50 nM (nanomolar); preferably at least 30 nM. As will be apparent to the skilled artisan, lower or higher plasma concentrations of the TGF-β response element decoy may be preferred depending on the mode of administration. For example, plasma concentrations of the TGF-β response element decoy of at least 30 nM can be appropriate in connection with intravenous, subcutaneous, intramuscular, controlled release, and oral administration methods, to name a few. In another example, relatively low circulating plasma levels of the TGF-β response element decoy can be desirable, however, when using local administration methods such as, for example, intratumor administration, intraocular administration, or implantation, which nevertheless can produce locally high, clinically effective concentrations of TGF-β response element decoy.

In yet another embodiment, the circulating plasma concentration of at least 20, 25, 30, 35, 40, 45, or 50 nM (nanomolar), preferably at least 30 nM, of the TGF-β response element decoy is achieved about 1, 2, 3, 4, 5, 6, 7, 8, 9 or 10 hours after the administration of the TGF-β response element decoy. In yet another embodiment, the circulating plasma concentration of at least 20, 25, 30, 35, 40, 45, or 50 nM (nanomolar), preferably at least 30 nM, of the TGF-β response element decoy is achieved in about 36 to 48 hours, preferably 24 to 35 hours, more preferably in 12 to 24 hours; most preferably in under 12 hours.

In a specific embodiment, the dose of a TGF-β response element decoy is a high dose. In one embodiment, the circulating plasma concentration of the TGF-β response element decoy is at least 30 nM.

In another embodiment, the circulating level of TGF-β response element decoy is 1 μM to 10 μM. In yet another embodiment, the circulating level of TGF-β response element decoy is 1, 2, 3, 4, 5, 6, 7, 8, 9 or 10 μM. In yet another embodiment, the circulating level of TGF-β response element decoy of 1 μM to 10 μM is achieved in about 36 to 48 hours, preferably 24 to 35 hours, more preferably in 12 to 24 hours; most preferably in under 12 hours.

The high dose may be achieved by several administrations per cycle. Alternatively, the high dose may be administered in a single bolus administration. A single administration of a high dose may result in circulating plasma levels of TGF-β response element decoy that are transiently much higher than 30 nM. Moreover, single administrations of particularly high doses of a TGF-β response element decoy may result in a circulating plasma concentration of TGF-β response element decoy of 1 μM to 10 μM in much less 12 hours, even in less than one hour.

Additionally, the dose of a TGF-β response element decoy may vary according to the particular TGF-β response element decoy used. The dose employed is likely to reflect a balancing of considerations, among which are stability, localization, cellular uptake, and toxicity of a particular TGF-β response element decoy. For example, a particular chemically modified TGF-β response element decoy may exhibit greater resistance to degradation, or may exhibit higher affinity for the target nucleic acid or protein, or may exhibit increased uptake by the cell or cell nucleus; all of which may permit the use of low doses. In another example, a particular chemically modified TGF-β response element decoy may exhibit lower toxicity than other oligomers, and therefore can be used at high doses. Thus, for a given TGF-β response element decoy, an appropriate dose to administer can be relatively high or relatively low. Appropriate doses or ranges of dosages would be appreciated by the skilled artisan, and the invention contemplates the continued assessment of optimal treatment schedules for particular species of TGF-β response element decoy. The daily dose can be administered in one or more treatments.

Other factors to be considered in determining an effective dose of a TGF-β response element decoy include whether the response element decoys will be administered in combination with other therapeutics. Treatment with a high dose of TGF-β response element decoy can result in combination therapies with reduced doses of additional therapeutics. In a specific embodiment, treatment with a particularly high dose of TGF-β response element decoy can result in combination therapies with greatly reduced doses of additional cancer therapeutics. For example, treatment of a patient with 10, 20, 30, 40, or 50 mg/kg/day of a TGF-β response element decoy can further increase the sensitivity of a subject to additional cancer therapeutics. In such cases, the particularly high dose of TGF-β response element decoy is combined with, for example, a greatly shortened radiotherapy schedule. In another example, the particularly high dose of a TGF-β response element decoy produces significant enhancement of the potency of additional cancer therapeutic agents.

Additionally, the particularly high doses of TGF-β response element decoy may further shorten the period of administration of a therapeutically effective amount of TGF-β response element decoy and/or additional cancer therapeutic agent, such that the length of a treatment cycle is much shorter than 14 days.

In one embodiment, TGF-β response element decoy is administered for 2 to 13 days at a dose of 0.01 to 10 mg/kg/day. In a specific embodiment, TGF-β response element decoy is administered for 2 to 3, 4 to 5, 6 to 7, 8 to 9, 10 to 11, or 12 to 13 days at a dose of0.01 to 1, 1 to 2, 3 to 4, 5 to 6,6 to 7, 7 to 8, or 9 to 10 mg/kg/day; more preferably at a dose of 4 to 9 mg/kg/day, and most preferably at a dose of 5 to 7 mg/kg/day. In another embodiment, TGF-β response element decoy is administered at said dose for 3 to 9 days. In yet another embodiment, TGF-β response element decoy is administered at said dose for 4 to 7 days. In a preferred embodiment, TGF-β response element decoy is administered at said dose for 5 to 6 days. In a most preferred embodiment, TGF-β response element decoy is administered at a dose of 5 to 7 mg/kg/day for 5 to 6 days. The invention contemplates other preferred treatment regimens depending on the particular TGF-β response element decoy to be used, or depending on the particular mode of administration, or depending on whether the TGF-β response element decoy is administered in combination with additional cancer therapeutic agents. The daily dose can be administered in one or more treatments.

In another embodiment, TGF-β response element decoy is administered at a particularly high dose of about 10 to 50 mg/kg/day. In a specific embodiment, TGF-β response element decoy is administered at a particularly high dose of about 10 to 15, 16 to 20,21 to 25,26 to 30, 31 to 35, 36 to 40,41 to 45, or 46 to 50 mg/kg/day. In a further embodiment, TGF-β response element decoy is administered at said dose for 1 to 10 days. In yet another embodiment, TGF-β response element decoy is administered at said dose for 2 to 7 days. In a yet another embodiment, TGF-β response element decoy is administered at said dose for 3 to 4 days. In a preferred embodiment, TGF-β response element decoy is administered at a dose of 10 to 15, 16 to 20, 21 to 25, 26 to 30, 31 to 35, 36 to 40, 41 to 45, or 46 to 50 mg/kg/day for a minimum of one day. The invention contemplates other preferred treatment regimens depending on the particular TGF-β response element decoy to be used, or depending on the particular mode of administration, or depending on whether the TGF-β response element decoy is administered in combination with additional cancer therapeutic agents. The daily dose can be administered in one or more treatments.

In one embodiment, the TGF-β response element decoy is administered alone. In another embodiment, a TGF-β response element decoy is administered in combination with a cancer therapeutic agent.

The invention contemplates the use or one of more TGF-β response element decoys. In one embodiment, the TGF-β response element decoy is administered prior to, subsequently, or concurrently with one or more TGF-β response element decoys, and/or additional cancer therapeutic agents for the prevention or treatment of cancer.

Combination therapy. Treatment of diseases such as cancer may benefit from combination therapy. For example, in hepatocellular cancer, treatment of the carcinogenesis cascade may be accomplished by inhibiting the inflammatory phase by nonsteroidal anti-inflammatory drugs which specifically inhibit the Cox-2 enzyme. The initial viral injection which causes the tissue injury can be treated with interferon therapy. Finally therapy can be directed at the proliferative phase to inhibit fibrosis by specifically inhibiting Type I collagen synthesis.

The Cox-2 enzyme is a mediator of inflammation and catalyzes the conversion of arachidonic acid to prostaglandins which inhibit apoptosis and tumor invasiveness (Fosslien, 2000, Crit. Rev. Clin. Lab. Sci. 37:431-502). Most human tumors overexpress Cox-2 but not Cox-1. Gene knockout transfection experiments demonstrate a central role of Cox-2 in experimental tumorigenesis. Nonsteroidal anti-inflammatory drugs which are selective inhibitors of the Cox-2 enzyme, such as Meloxicam, Celecoxib (SC-58635) and Rofecoxib (MK-0966) reduce prostaglandin synthesis, restore apoptosis and inhibit cancer cell proliferation (Fosslien, 2000, Crit. Rev. Clin. Lab. Sci. 37:431-502, Giercksky, 2001, Best Pract. Res. Clin. Gastroenterol. 15:821-833). The overexpression of Cox-2 in many premalignant, malignant and metastatic human cancers has been shown to significantly correlate to invasiveness, prognosis and survival in some cancers (Koki et al., 2002, Cancer Control. 9:28-35).

Cox-2-derived prostaglandins contribute to tumor growth by inducing neoangiogenesis that sustains tumor cell viability and growth (Masferrer et al., 2000, Cancer Res. 60:1306-1311). Cox-2 is expressed within the human tumor neovasculature as well as in neoplastic cells present in human colon, breast, prostate and lung as well as many other cancers including pancreatic (Kokawa et al., 2001, Cancer 91:333), esophageal (Shirvani et al., 2000, Gastroenterol. 118:487), bladder (Yoshimura et al., 2001, J. Urology 165:1468), gastric (Ohno et al., 2001, Cancer 91:1876), lung (Brabender et al., 2002, Ann. Surgery 235:440), colon (Cianchi et al., 2001, Gastroenterol. 121:1339), breast (Soslow et al., 2000, Cancer 89:2637), head & neck squamous cell carcinomas (Jaeckel et al., 2001, Arch. Otolaryngology Head & Neck Surg. 127:1253), prostate (Yoshimura et al., 2000, Cancer 89:589), retinoblastoma (Karim et al., 2000, J. Ophthalmol. 129:398), thyroid (Nose et al., 2002, Am. J. Clin. Pathol. 117:546), ovarian (Denkert et al., 2002, Am. J. Pathol. 160:893), cervix (Sales et al., 2001, J. Clin. Endocrinol & Metab. 86:2243), endometria (Ferranthna et al., 2002, Cancer 95:801). Many of these cancers can be successively treated with nonsteroidal anti-inflammatory drugs specifically directed against the Cox-2 enzyme including NS-398 for hepatocellular carcinoma (Cheng et al., 2002, Int. J. Cancer 99:755), prostate (Liu et al., 2000, J. Urology 164:820), and leukemia (Nakanishi et al., 2001, Cancer Res. 61:1451), SC-236 for breast (Connolly et al., 2002, Brit. J. Cancer 87:231) and brain (Portnow et al., 2002, Neuro-Oncol. 4:22), celecoxib for colon (Reddy et al., 2000, Cancer Res. 60:293), skin (Orengo et al., 2002, Arch. Dermatol. 138:751), oral (Wang et al., 2002, Larybgnscope 112:839), bladder (Grubbs et al., 2000, Cancer Res. 60:5599), and neuroectodermal (Patti et al., 2002, Cancer Lett., 189:13), nabumetone for leukemia (Nakanishi et al., 2001, Cancer Res. 61:1451) and nimesulide for lung (Hida et al., 2000, Clin. Cancer Res. 6:2006) and tongue (Shiotani et al., 2001, Cancer Res. 61:1451). Furthermore, the antitumorigenic effectiveness of Cox-2 inhibitors can be enhanced by radiotherapy without harmful effects to the normal surrounding tissue (IKishi et al., 2000, Cancer Res. 60:1326-1331).

Although the Cox-2 enzyme may play a key role in the early stages of HCC, it does not play a key role in the advanced stages of the disease and therefore may be consequently related to HCC dedifferentiation (Koga et al., 1999, Hepatology 29:688-696; Shiota et al., 1999, Hepato-Gastroenterol. 46:407-412). In HCC, the combined expression of inducible nitric oxide synthase and Cox-2 play an important role in prognosis of hepatitis C virus-positive HCC patients which could be partially attributable to the modulation of angiogenesis by the Cox-2 enzyme (Rahman et al., 2001, Clin. Cancer Res. 7:1325-1332). The down-regulation of the novel tumor suppressor (PTEN) is an important step in hepatitis C virus-positive cirrhotic HCC and may result in concomitant up-regulation of inducible nitric oxide synthase and Cox-2 in the surrounding liver in favor of tumor promotion (Rahman et al., 2002, Intl. J. Cancer 100:152-157).

The early stage of HCC is associated with increased DNA methyltransferase expression. Since this is an early event, this enzyme is a potential target for HCC preventive therapy (Sun et al., 1997, Jpn. J. Cancer Res 88:1165-1170). Another study using comparative genomic hybridization demonstrate a possible link between HCC tumor size and the burden of genetic changes (Kitay-Cohen et al., 2001, Cancer Genet. Cytogenet. 131:60-64).

The predominant reason for undergoing liver transplantation is hepatitis C infection which is the most common basis for liver disease in the United States (Alter et al., 2000, Seminar Liver Dis. 20:17-35). The disease it causes is characterized by a high rate of viral persistence and the development of chronic liver disease leading to cirrhosis and then to HCC. There is a slow sequential progression from hepatitis C viral infection through cirrhosis and HCC (Castells et al., 1995, Liver 15:159-163). A high serum level of hepatocyte growth factor reveals a high carcinogenic status in chronic hepatitis and liver cirrhosis induced by the C type virus (Yamagami et al., 2001, Intervirology 44:36-42).

Alcohol intake is a high risk factor in chronic hepatitis C viral infection (Khan et al., 2000, Alcohol & Alcoholism 35:286-295; Hellerbrand et al., 2001, Digest. Dis. 19:345-351). Alcohol consumption is an important risk factor which has no relation to hepatitis C virus replication. Chronic hepatitis C virus carriers should avoid excessive alcohol intake to reduce the acceleration of liver disease and the risk of HCC. Considering the poor prognosis of HCC associated with chronic alcohol abuse, prevention is of pivotal importance.

Interferon therapy reduces the risk of HCC, especially among virologic or biochemical responders (Yoshida et al., 1999, Ann. Internal Med. 131:174-181). However, although it appears that interferon therapy is beneficial in the prevention of HCC in patients with viral hepatitis, more experience is required (Franco et al., 2002, J. Vasc. Interv. Radiol. 13:S191-S196). Interferon therapy does have long-term beneficial effects in terms of B virus clearance, reduction in HCC and prolonging survival (Lin et al., 1999, Hepatology 29:971-975; Poynard et al., 1999, Clin. Liver Dis. 3:869-881). In a retrospective analysis of 652 patients with chronic hepatitis C who had been treated with interferon, the therapy produced an improvement in the histological activity and the fibrosis stage in the second biopsy specimens irrespective of the clinical outcome when compared against untreated subjects (Takimoto et al., 2002, Digest. Dis. Sci. 47:170-176).

In a preferred embodiment, the invention further encompasses the use of combination therapy to prevent or treat a disease or condition associated with aberrant expression of a gene regulated by TGF-β including, but not limited to, fibrotic diseases and cancer. For example, hepatocellular cancer can be treated with a pharmaceutical composition comprising a TGF-β response element decoy in combination with a glucocorticosteroid.

Combination therapy also includes, in addition to administration of a TGF-β response element decoy, the use of one or more molecules, compounds or treatments that aid in the prevention or treatment of fibrotic diseases, which molecules, compounds or treatments includes, but is not limited to, chemoagents, immunotherapeutics, vaccines, anti angiogenic agents, cytokines, hormone therapies, gene therapies, and radiotherapies.

In one embodiment, the invention further encompasses the use of combination therapy to prevent or treat cancer. For example, prostate cancer can be treated with a pharmaceutical composition comprising a TGF-β response element decoy in combination with paclitaxel, docetaxel, mitoxantrone, and/or an androgen receptor antagonist (e.g., flutamide). As another example, breast cancer can be treated with a pharmaceutical composition comprising a TGF-β response element decoy in combination with docetaxel, paclitaxel, cisplatin, 5 fluorouracil, doxorubicin, and/or VP 16 (etoposide). As another example, leukemia can be treated with a pharmaceutical composition comprising a TGF-β response element decoy in combination with fludarabine, cytosine arabinoside, gemtuzumab (MYLOTARG), daunorubicin, methotrexate, vincristine, 6 mercaptopurine, idarubicin, mitoxantrone, etoposide, asparaginase, prednisone and/or cyclophosphamide. As another example, myeloma can be treated with a pharmaceutical composition comprising a TGF-β response element decoy in combination with dexamethasone. As another example, melanoma can be treated with a pharmaceutical composition comprising a TGF-β response element decoy in combination with dacarbazine. As another example, colorectal cancer can be treated with a pharmaceutical composition comprising a TGF-β response element decoy in combination with irinotecan. As another example, lung cancer can be treated with a pharmaceutical composition comprising a TGF-β response element decoy in combination with paclitaxel, docetaxel, etoposide and/or cisplatin. As another example, non Hodgkin's lymphoma can be treated with a pharmaceutical composition comprising a TGF-β response element decoy in combination with cyclophosphamide, CHOP, etoposide, bleomycin, mitoxantrone and/or cisplatin. As another example, gastric cancer can be treated with a pharmaceutical composition comprising a TGF-β response element decoy in combination with cisplatin. As another example, pancreatic cancer can be treated with a pharmaceutical composition comprising a TGF-β response element decoy in combination with gemcitabine. These combination therapies can also be used to prevent cancer or the recurrence of cancer.

Combination therapy also includes, in addition to administration of a TGF-β response element decoy, the use of one or more molecules, compounds or treatments that aid in the prevention or treatment of cancer, which molecules, compounds or treatments includes, but is not limited to, chemoagents, immunotherapeutics, cancer vaccines, anti angiogenic agents, cytokines, hormone therapies, gene therapies, and radiotherapies.

In one embodiment, one or more chemoagents, in addition to a TGF-β response element decoy, is administered to treat a cancer patient. Examples of chemoagents contemplated by the present invention include, but are not limited to, cytosine arabinoside, taxoids (e.g., paclitaxel, docetaxel), anti tubulin agents (e.g., paclitaxel, docetaxel, Epothilone B, or its analogues), cisplatin, carboplatin, adriamycin, tenoposide, mitozantron, 2-chlorodeoxyadenosine, alkylating agents (e.g., cyclophosphamide, mechlorethamine, thioepa, chlorambucil, melphalan, carmustine (BSNU), lomustine (CCNU), cyclothosphamide, busulfan, dibromomannitol, streptozotocin, mitomycin C, and cis dichlorodiamine platinum (II) (DDP) cisplatin, thio tepa), antibiotics (e.g., dactinomycin (formerly actinomycin), bleomycin, mithramycin, anthramycin), antimetabolites (e.g., methotrexate, 6 mercaptopurine, 6 thioguanine, cytarabine, 5 fluorouracil, fludarabine, gemcitabine, dacarbazine, temozolamide), asparaginase, Bacillus Calmette and Guerin, diphtheria toxin, hexamethylmelamine, hydroxyurea, LYSODREN®, nucleoside analogues, plant alkaloids (e.g., Taxol, paclitaxel, camptothecin, topotecan, irinotecan (CAMPTOSAR, CPT 11), vincristine, vinca alkyloids such as vinblastine), podophyllotoxin (including derivatives such as epipodophyllotoxin, VP 16 (etoposide), VM 26 (teniposide)), cytochalasin B, gramicidin D, ethidium bromide, emetine, mitomycin, procarbazine, mechlorethamine, anthracyclines (e.g., daunorubicin (formerly daunomycin), doxorubicin, doxorubicin liposomal), dihydroxyanthracindione, mitoxantrone, mithramycin, actinomycin D, procaine, tetracaine, lidocaine, propranolol, puromycin, anti mitotic agents, abrin, ricin A, pseudomonas exotoxin, nerve growth factor, platelet derived growth factor, tissue plasminogen activator, aldesleukin, allutamine, anastrozle, bicalutamide, biaomycin, busulfan, capecitabine, carboplain, chlorabusil, cladribine, cylarabine, daclinomycin, estramusine, floxuridhe, gamcitabine, gosereine, idarubicin, itosfamide, lauprolide acetate, levamisole, lomusline, mechlorethamine, magestrol, acetate, mercaptopurino, mesna, mitolanc, pegaspergase, pentoslatin, picamycin, riuxlmab, campath 1, straplozocin, thioguanine, tretinoin, vinorelbine, or any fragments, family members, or derivatives thereof, including pharmaceutically acceptable salts thereof. Compositions comprising one or more chemoagents (e.g., FLAG, CHOP) are also contemplated by the present invention. FLAG comprises fludarabine, cytosine arabinoside (Ara C) and G CSF. CHOP comprises cyclophosphamide, vincristine, doxorubicin, and prednisone.

In one embodiment, said chemoagent is dacarbazine at a dose ranging from 200 to 4000 mg/m²/cycle. In a preferred embodiment, said dose ranges from 700 to 1000 mg/m²/cycle. In another embodiment, said chemoagent is fludarabine at a dose ranging from 25 to 50 mg/m²/cycle. In another embodiment, said chemoagent is cytosine arabinoside (Ara C) at a dose ranging from 200 to 2000 mg/m²/cycle. In another embodiment, said chemoagent is docetaxel at a dose ranging from 1.5 to 7.5 mg/kg/cycle. In another embodiment, said chemoagent is paclitaxel at a dose ranging from 5 to 15 mg/kg/cycle. In yet another embodiment, said chemoagent is cisplatin at a dose ranging from 5 to 20 mg/kg/cycle. In yet another embodiment, said chemoagent is 5 fluorouracil at a dose ranging from 5 to 20 mg/kg/cycle. In yet another embodiment, said chemoagent is doxorubicin at a dose ranging from 2 to 8 mg/kg/cycle. In yet another embodiment, said chemoagent is epipodophyflotoxin at a dose ranging from 40 to 160 mg/kg/cycle. In yet another embodiment, said chemoagent is cyclophosphamide at a dose ranging from 50 to 200 mg/kg/cycle. In yet another embodiment, said chemoagent is irinotecan at a dose ranging from 50 to 75, 75 to 100, 100 to 125, or 125 to 150 mg/m²/cycle. In yet another embodiment, said chemoagent is vinblastine at a dose ranging from 3.7 to 5.4, 5.5 to 7.4, 7.5 to 11, or 11 to 18.5 mg/m²/cycle. In yet another embodiment, said chemoagent is vincristine at a dose ranging from 0.7 to 1.4, or 1.5 to 2 mg/m²/cycle. In yet another embodiment, said chemoagent is methotrexate at a dose ranging from 3.3 to 5, 5 to 10, 10 to 100, or 100 to 1000 mg/m²/cycle.

In a preferred embodiment, the invention further encompasses the use of low doses of chemoagents when administered as part of a TGF-β response element decoy treatment regimen. For example, initial treatment with a TGF-β response element decoy increases the sensitivity of a tumor to subsequent challenge with a dose of chemoagent, which dose is near or below the lower range of dosages when the chemoagent is administered without the oligomer. In one embodiment, a TGF-β response element decoy and a low dose (e.g., 6 to 60 mg/m²day or less) of docetaxel are administered to a cancer patient. In another embodiment, a TGF-β response element decoy and a low dose (e.g., 10 to 135 mg/m²/day or less) of paclitaxel are administered to a cancer patient. In yet another embodiment, a TGF-β response element decoy and a low dose (e.g., 2.5 to 25 mg/m²/day or less) of fludarabine are administered to a cancer patient. In yet another embodiment, a TGF-β response element decoy and a low dose (e.g., 0.5 to 1.5 g/m²/day or less) of cytosine arabinoside (Ara C) are administered to a cancer patient.

The invention, therefore, contemplates the use of a TGF-β response element decoy, which is administered prior to, subsequently, or concurrently with low doses of chemoagents, for the prevention or treatment of cancer.

In one embodiment, said chemoagent is cisplatin, e.g., PLATINOL or PLATINOL AQ (Bristol Myers), at a dose ranging from 5 to 10, 10 to 20, 20 to 40, or 40 to 75 mg/m²/cycle. In another embodiment, a dose of cisplatin ranging from 7.5 to 75 mg/m²/cycle is administered to a patient with ovarian cancer. In another embodiment, a dose of cisplatin ranging from 5 to 50 mg/m²/cycle is administered to a patient with bladder cancer.

In another embodiment, said chemoagent is carboplatin, e.g., PARAPLATIN (Bristol Myers), at a dose ranging from 2 to 4, 4 to 8, 8 to 16, 16 to 35, or 35 to 75 mg/m²/cycle. In another embodiment, a dose of carboplatin ranging from 7.5 to 75 mg/m2/cycle is administered to a patient with ovarian cancer. In another embodiment, a dose of carboplatin ranging from 5 to 50 mg/m²/cycle is administered to a patient with bladder cancer. In another embodiment, a dose of carboplatin ranging from 2 to 20 mg/m²/cycle is administered to a patient with testicular cancer.

In another embodiment, said chemoagent is cyclophosphamide, e.g., CYTOXAN (Bristol Myers Squibb), at a dose ranging from 0.25 to 0.5, 0.5 to 1, 1 to 2, 2 to 5, 5 to 10, 10 to 20, 20 to 40 mg/kg/cycle. In another embodiment, a dose of cyclophosphamide ranging from 4 to 40 mg/kg/cycle is administered to a patient with malignant cancer. In another embodiment, a dose of cyclophosphamide ranging from 0.25 to 2.5 mg/kg/cycle is administered to a patient with non malignant cancer.

In one embodiment, said chemoagent is cytarabine, e.g., CYTOSAR U (Pharmacia & Upjohn), at a dose ranging from 0.5 to 1, 1 to 4, 4 to 10, 10 to 25, 25 to 50, or 50 to 100 mg/m²/cycle. In another embodiment, a dose of cytarabine ranging from 10 to 100 mg/m²/cycle is administered to a patient with acute leukemia. In another embodiment, a dose of cytarabine ranging from 0.5 to 5 mg/m²/cycle is administered to a patient with meningeal leukemia. In another embodiment, a dose of cytarabine liposome, e.g., DEPOCYT (Chiron Corp.) ranging from 5 to 50 mg/m²/cycle is administered to a patient with cancer.

In another embodiment, said chemoagent is dacarbazine, e.g., DTIC or DTIC DOME (Bayer Corp.), at a dose ranging from 15 to 250 mg/m²/cycle or ranging from 0.2 to 2 mg/kg/cycle. In another embodiment, a dose of dacarbazine ranging from 15 to 150 mg/m²/cycle is administered to a patient with Hodgkin's disease. In another embodiment, a dose of dacarbazine ranging from 0.2 to 2 mg/kg/cycle is administered to a patient with malignant melanoma.

In another embodiment, said chemoagent is topotecan, e.g., HYCAMTIN (SmithKline Beecham), at a dose ranging from 0.1 to 0.2, 0.2 to 0.4, 0.4 to 0.8, or 0.8 to 1.5 mg/m²/cycle.

In another embodiment, said chemoagent is irinotecan, e.g., CAMPTOSAR (Pharmacia & Upjohn), at a dose ranging from 5 to 10, 10 to 25, or 25 to 50 mg/m²/cycle.

In another embodiment, said chemoagent is fludarabine, e.g., FLUDARA (Berlex Laboratories), at a dose ranging from 2.5 to 5, 5 to 10, 10 to 15, or 15 to 25 mg/m²/cycle.

In another embodiment, said chemoagent is cytosine arabinoside (Ara C) at a dose ranging from 200 to 2000 mg/m²/cycle.

In another embodiment, said chemoagent is docetaxel, e.g., TAXOTERE (Rhone Poulenc Rorer) at a dose ranging from 6 to 10, 10 to 30, or 30 to 60 mg/m²/cycle.

In another embodiment, said chemoagent is paclitaxel, e.g., TAXOL (Bristol Myers Squibb), at a dose ranging from 10 to 20, 20 to 40, 40 to 70, or 70 to 135 mg/kg/cycle.

In another embodiment, said chemoagent is 5 fluorouracil at a dose ranging from 0.5 to 5 mg/kg/cycle.

In another embodiment, said chemoagent is doxorubicin, e.g., ADRIAMYCIN (Pharmacia & Upjohn), DOXIL (Alza), RUBEX (Bristol Myers Squibb), at a dose ranging from 2 to 4, 4 to 8, 8 to 15, 15 to 30, or 30 to 60 mg/kg/cycle.

In another embodiment, said chemoagent is etoposide, e.g., VEPESID (Pharmacia & Upjohn), at a dose ranging from 3.5 to 7, 7 to 15, 15 to 25, or 25 to 50 mg/m²/cycle. In another embodiment, a dose of etoposide ranging from 5 to 50 mg/m²/cycle is administered to a patient with testicular cancer. In another embodiment, a dose of etoposide ranging from 3.5 to 35 mg/m²/cycle is administered to a patient with small cell lung cancer.

In another embodiment, said chemoagent is vinblastine, e.g., VELBAN (Eli Lilly), at a dose ranging from 0.3 to 0.5, 0.5 to 1, 1 to 2, 2 to 3, or 3 to 3.7 mg/m²/cycle.

In another embodiment, said chemoagent is vincristine, e.g., ONCOVIN (Eli Lilly), at a dose ranging from 0.1, 0.2, 0.3, 0.4, 0.5, 0.6 or 0.7 mg/m²/cycle.

In another embodiment, said chemoagent is methotrexate at a dose ranging from 0.2 to 0.9, 1 to 5, 5 to 10, 10 to 20.

In a preferred embodiment, the invention further encompasses the use of low doses of therapeutic agents when administered as part of a TGF-β response element decoy treatment regimen. For example, initial treatment with a TGF-β response element decoy increases the sensitivity of a disease to subsequent challenge with a dose of a therapeutic agent, which dose is near or below the lower range of dosages when the therapeutic agent is administered without the response element decoy.

The invention, therefore, contemplates the use of a TGF-β response element decoy which is administered prior to, subsequently, or concurrently with low doses of therapeutic agents, for the prevention or treatment of fibrotic disease.

In another embodiment, a TGF-β response element decoy is administered in combination with one or more immunotherapeutic agents, such as antibodies and immunomodulators, which includes, but is not limited to, rituxan, rituximab, campath 1, gemtuzumab, or trastuzumab.

In another embodiment, a TGF-β response element decoy is administered in combination with one or more antiangiogenic agents, which includes, but is not limited to, angiostatin, thalidomide, kringle 5, endostatin, Serpin (Serine Protease Inhibitor) anti thrombin, 29 kDa N-terminal and a 40 kDa C terminal proteolytic fragments of fibronectin, 16 kDa proteolytic fragment of prolactin, 7.8 kDa proteolytic fragment of platelet factor 4, a 13 amino acid peptide corresponding to a fragment of platelet factor 4 (Maione et al., 1990, Cancer Res. 51:2077 2083), a 14 amino acid peptide corresponding to a fragment of collagen I (Tolma et al., 1993, J. Cell Biol. 122:497 511), a 19 amino acid peptide corresponding to a fragment of Thrombospondin I (Tolsma et al., 1993, J. Cell Biol. 122:497 511), a 20 amino acid peptide corresponding to a fragment of SPARC (Sage et al., 1995, J. Cell. Biochem. 57:1329 1334), or any fragments, family members, or derivatives thereof, including pharmaceutically acceptable salts thereof.

Other peptides that inhibit angiogenesis and correspond to fragments of laminin, fibronectin, procollagen, and EGF have also been described (see the review by Cao, 1998, Prog. Mol. Subcell. Biol. 20:161 176). Monoclonal antibodies and cyclic pentapeptides, which block certain integrins that bind RGD proteins (i.e., possess the peptide motif Arg Gly Asp), have been demonstrated to have anti vascularization activities (Brooks et al., 1994, Science 264:569 571; Hammes et al., 1996, Nature Medicine 2:529 533). Moreover, inhibition of the urokinase plasminogen activator receptor by receptor antagonists inhibits angiogenesis, tumor growth and metastasis (Min et al., 1996, Cancer Res. 56: 2428 33; Crowley et al., 1993, Proc. Natl. Acad. Sci. USA 90:5021 25). Use of such antiangiogenic agents is also contemplated by the present invention.

In another embodiment, a TGF-β response element decoy is administered in combination with a regimen of radiation.

In another embodiment, a TGF-β response element decoy is administered in combination with one or more cytokines, which includes, but is not limited to, interferons, lymphokines, tumor necrosis factors, tumor necrosis factor like cytokines, lymphotoxin α, lymphotoxin β, interferon α, interferon β, macrophage inflammatory proteins, granulocyte monocyte colony stimulating factor, interleukins (including, but not limited to, interleukin 1, interleukin 2, interleukin 6, interleukin 12, interleukin 15, interleukin 18), OX40, CD27, CD30, CD40 or CD137 ligands, Fas-Fas ligand, 4 1BBL, endothelial monocyte activating protein or any fragments, family members, or derivatives thereof, including pharmaceutically acceptable salts thereof

In another embodiment, a TGF-β response element decoy is administered in combination with one or more growth factors.

In yet another embodiment, a TGF-β response element decoy is administered in combination with a cancer vaccine. Examples of cancer vaccines include, but are not limited to, autologous cells or tissues, non autologous cells or tissues, carcinoembryonic antigen, alpha fetoprotein, human chorionic gonadotropin, BCG live vaccine, melanocyte lineage proteins (e.g., gp100, MART 1/MelanA, TRP 1 (gp75), tyrosinase, widely shared tumor specific antigens (e.g., BAGE, GAGE 1, GAGE 2, MAGE 1, MAGE 3, N acetylglucosaminyltransferase V, p15), mutated antigens that are tumor specific (β catenin, MUM 1, CDK4), nonmelanoma antigens (e.g., HER 2/neu (breast and ovarian carcinoma), human papillomavirus E6, E7 (cervical carcinoma), MUC 1 (breast, ovarian and pancreatic carcinoma)). For human tumor antigens recognized by T cells, see generally Robbins et al., 1996, Curr. Opin. Immunol. 8:628 36. Cancer vaccines may or may not be purified preparations.

In yet another embodiment, a TGF-β response element decoy is administered in combination with a hepatitis vaccine.

In yet another embodiment, a TGF-β response element decoy is used in association with a hormonal treatment. Hormonal therapeutic treatments comprise hormonal agonists, hormonal antagonists (e.g., flutamide, tamoxifen, leuprolide acetate (LUPRON)), and steroids (e.g., dexamethasone, retinoids, betamethasone, cortisol, cortisone, prednisone, dehydrotestosterone, glucocorticoids, mineralocorticoids, estrogen, testosterone, progestins).

In yet another embodiment, a TGF-β response element decoy is used in association with a gene therapy program.

Pharmaceutical Compositions. The present invention further provides for a pharmaceutical composition that comprises a TGF-β response element decoy, and a pharmaceutically acceptable carrier. Suitable pharmaceutically acceptable carriers include essentially chemically inert and nontoxic pharmaceutical compositions that do not interfere with the effectiveness of the biological activity of the pharmaceutical composition. Examples of suitable pharmaceutical carriers include, but are not limited to, saline solutions, glycerol solutions, ethanol, N (1(2,3 dioleyloxy)propyl) N,N,N trimethylammonium chloride (DOTMA), diolesylphosphotidylethanolamine (DOPE), and liposomes. Oligonucleotide encapsulation in liposomes is described in U.S. Pat. No. 5,665,710, entitled “Method of making liposomal oligodeoxynucleotide compositions”, issued Sep. 9, 1997, herein incorporated by reference in its entirety. Such pharmaceutical compositions should contain a therapeutically effective amount of the compound, together with a suitable amount of carrier so as to provide the form for proper administration to the patient. The formulation should suit the mode of administration. For example, oral administration requires enteric coatings to protect the response element decoys from degradation within the gastrointestinal tract. In another example, the response element decoys may be administered in a liposomal formulation to shield the response element decoys from degradative enzymes, facilitate transport in circulatory system, and effect delivery across cell membranes to intracellular sites.

In another embodiment, a pharmaceutical composition comprises a TGF-β response element decoy, and/or one or more therapeutic agents (as described herein); and a pharmaceutically acceptable carrier. In a particular embodiment, the pharmaceutical composition comprises a TGF-β response element decoy and one or more cancer therapeutic agents; and a pharmaceutically acceptable carrier.

In one embodiment, a pharmacutical composition, comprising a TGF-β response element decoy, with or without other therapeutic agents; and a pharmaceutically acceptable carrier, is at an effective dose.

The invention further provides a pharmaceutical kit comprising an effective amount of a TGF-β response element decoy, to prevent or treat a disease associated with tissue fibrosis or cancer. In a specific embodiment, the TGF-β response element decoy comprises the sequence 5′-AGCCTAACTGCCCACGGCCAGCGACGT-3′ (SEQ ID NO: 2). The kit may comprise one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, the pharmaceutical composition comprises a TGF-β response element decoy at a dose of about 0.01 to 0.1, 0.1 to 1, 1 to 5, or 6 to 10 mg/kg/day; preferably at a dose of 4 to 9 mg/kg/day; more preferably at a dose of 5 to 7 mg/kg/day; and a pharmaceutically acceptable carrier. In another embodiment, the pharmaceutical composition comprises a TGF-β response element decoy at a dose of 0.01, 0.025, 0.05, 0.075, 1, 2, 3, 4, 5, 6, 7, 8, 9 or 10 mg/kg/day; and a pharmaceutically acceptable carrier. The actual amount of any particular response element decoy administered can depend on several factors, such as the type of cancer, the toxicity of the response element decoy to normal cells of the body, the rate of uptake of the response element decoy by tumor cells, and the weight and age of the individual to whom the response element decoy is administered. Because of the many factors present in vivo that may interfere with the action or biological activity of the response element decoy, an effective amount of the response element decoy may vary for each individual.

In another embodiment, the pharmaceutical compositions of the invention comprise a particularly high dose, which ranges from about 10 to 50 mg/kg/day. In a specific embodiment, a particularly high dose of TGF-β response element decoy, ranging from 11 to 20, 21 to 30, 31 to 40, or 41 to 50 mg/kg/day, is administered during a treatment cycle. In another specific embodiment, the particularly high dose of TGF-β response element decoy, ranges from 11 to 15, 16 to 20, 21 to 25, 26 to 30, 31 to 35, 36 to 40, 41 to 45, or 46 to 50 mg/kg/day during a treatment cycle.

Selection of the preferred effective dose can be determined (e.g., via clinical trials) by a skilled artisan based upon the consideration of several factors which will be known to one of ordinary skill in the art. Such factors include the particular form of response element decoy, the response element decoy's pharmacokinetic parameters such as bioavailability, metabolism, half-life, etc., which is established during the development procedures typically employed in obtaining regulatory approval of a pharmaceutical compound. Further factors in considering the dose include the disease to be treated, the benefit to be achieved in a patient, the patient's body mass, the patient's immune status, the route of administration, whether administration of the response element decoy or combination therapeutic agent is acute or chronic, concomitant medications, and other factors known by the skilled artisan to affect the efficacy of administered pharmaceutical agents.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2 ethylamino ethanol, histidine, procaine, etc.

In a preferred embodiment, the composition is formulated in accordance with routine procedures as a pharmaceutical composition adapted for subcutaneous injection or intravenous administration to humans. Typically, pharmaceutical compositions for subcutaneous injection or intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the composition is to be administered by infusion, it can be dispensed with an infusion bottle, bag, or other acceptable container, containing sterile pharmaceutical grade water, saline, or other acceptable diluents. Where the composition is administered by injection, an ampule of sterile water for injection or saline can be provided so that the ingredients may be mixed prior to administration.

Modes of Administration. Administration of the pharmaceutical compositions of the invention includes, but is not limited to, oral, intravenous infusion, subcutaneous injection, intramuscular, topical, depo injection, implantation, time-release mode, intracavitary, intranasal, inhalation, intratumor, intraocular, and controlled release. The pharmaceutical compositions of the invention also may be introduced parenterally, transmucosally (e.g., orally), nasally, rectally, intravaginally, sublingually, submucosally, or transdermally. Preferably, administration is parenteral, i.e., not through the alimentary canal but rather through some other route via, for example, intravenous, subcutaneous, intramuscular, intraperitoneal, intraorbital, intracapsular, intraspinal, intrasternal, intra-arterial, or intradermal administration. The skilled artisan can appreciate the specific advantages and disadvantages to be considered in choosing a mode of administration. Multiple modes of administration are encompassed by the invention. For example, a TGF-β response element decoy is administered by subcutaneous injection, whereas a combination therapeutic agent is administered by intravenous infusion. Moreover, administration of one or more species of TGF-β response element decoys, with or without other therapeutic agents, may occur simultaneously (i.e., co-administration) or sequentially. For example, a TGF-β response element decoy is first administered to increase sensitivity of a tumor to subsequent administration of a therapeutic agent or irradiation therapy. In another embodiment, the periods of administration of one or more species of TGF-β response element decoy, with or without other therapeutic agents may overlap. For example, a TGF-β response element decoy is administered for 7 days, and a second therapeutic agent is introduced beginning on the fifth day of TGF-β response element decoy treatment, and treatment with the second therapeutic agent continues beyond the 7-day TGF-β response element decoy treatment.

Pharmaceutical compositions adapted for oral administration may be provided, for example, as capsules or tablets; as powders or granules; as solutions, syrups or suspensions (in aqueous or non-aqueous liquids); as edible foams or whips; or as emulsions. Tablets or hard gelatine capsules may comprise, for example, lactose, starch or derivatives thereof, magnesium stearate, sodium saccharine, cellulose, magnesium carbonate, stearic acid or salts thereof. Soft gelatine capsules may comprise, for example, vegetable oils, waxes, fats, semi-solid, or liquid polyols etc. Solutions and syrups may comprise, for example, water, polyols and sugars.

An active agent intended for oral administration may be coated with or admixed with a material (e.g., glyceryl monostearate or glyceryl distearate) that delays disintegration or affects absorption of the active agent in the gastrointestinal tract. Thus, for example, the sustained release of an active agent may be achieved over many hours and, if necessary, the active agent can be protected from being degraded within the gastrointestinal tract. Taking advantage of the various pH and enzymatic conditions along the gastrointestinal tract, pharmaceutical compositions for oral administration may be formulated to facilitate release of an active agent at a particular gastrointestinal location.

Pharmaceutical compositions adapted for parenteral administration include, but are not limited to, aqueous and non-aqueous sterile injectable solutions or suspensions, which may contain antioxidants, buffers, bacteriostats and solutes that render the pharmaceutical compositions substantially isotonic with the blood of an intended recipient. Other components that may be present in such pharmaceutical compositions include water, alcohols, polyols, glycerine and vegetable oils, for example. Compositions adapted for parenteral administration may be presented in unit-dose or multi-dose containers, for example, sealed ampules and vials, and may be stored in a freeze-dried (lyophilized) condition requiring the addition of a sterile liquid carrier, e.g., sterile saline solution for injections, immediately prior to use. Extemporaneous injection solutions and suspensions may be prepared from sterile powders, granules and tablets. Such pharmaceutical compositions should contain a therapeutically effective amount of a TGF-β response element decoy, together with a suitable amount of carrier so as to provide the form for proper administration to the patient. The formulation should suit the mode of administration.

Pharmaceutical compositions adapted for transdermal administration may be provided as discrete patches intended to remain in intimate contact with the epidermis for a prolonged period of time. Pharmaceutical compositions adapted for topical administration may be provided as, for example, ointments, creams, suspensions, lotions, powders, solutions, pastes, gels, sprays, aerosols or oils. A topical ointment or cream is preferably used for topical administration to the skin, mouth, eye or other external tissues. When formulated in an ointment, the active ingredient may be employed with either a paraffinic or a water-miscible ointment base. Alternatively, the active ingredient may be formulated in a cream with an oil-in-water base or a water-in-oil base.

Pharmaceutical compositions adapted for topical administration to the eye include, for example, eye drops or injectable pharmaceutical compositions. In these pharmaceutical compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilies and mouthwashes.

Pharmaceutical compositions adapted for nasal administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, pharmaceutical compositions adopted for nasal administration may comprise liquid carriers such as, for example, nasal sprays or nasal drops. These pharmaceutical compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient.

Pharmaceutical compositions adapted for rectal administration may be provided as suppositories or enemas. Pharmaceutical compositions adapted for vaginal administration may be provided, for example, as pessaries, tampons, creams, gels, pastes, foams or spray formulations.

In one embodiment, a pharmaceutical composition of the invention is delivered by a controlled-release system. For example, the pharmaceutical composition may be administered using intravenous infusion, an implantable osmotic pump, a transdermal patch, liposomes, or other modes of administration. In one embodiment, a pump may be used (See, e.g., Langer, 1990, Science 249:1527-33; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, the compound can be delivered in a vesicle, in particular a liposome (See, e.g., Langer, Science 249:1527-33 (1990); Treat et al., 1989, in Liposomes in the Therapy of Infectious Disease and Cancer, Lopez Berestein and Fidler (eds.), Liss, New York, pp. 353-65; Lopez-Berestein, ibid., pp. 317-27 International Patent Publication No. WO 91/04014; U.S. Pat. No. 4,704,355). In another embodiment, polymeric materials can be used (See, e.g., Medical Applications of Controlled Release, Langer and Wise (eds.), CRC Press: Boca Raton, Florida, 1974; Controlled Drug Bioavailability, Drug Product Design and Performance, Smolen and Ball (eds.), Wiley: New York (1984); Ranger and Peppas, 1953, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In yet another embodiment, a controlled release system can be placed in proximity of the target. For example, a micropump may deliver controlled doses directly into the brain, thereby requiring only a fraction of the systemic dose (See, e.g., Goodson, 1984, in Medical Applications of Controlled Release, vol. 2, pp. 115-138).

In one embodiment, it may be desirable to administer the pharmaceutical composition of the invention locally to the area in need of treatment; this may be achieved, for example, and not by way of limitation, by local infusion during surgery, topical application (e.g., in conjunction with a wound dressing after surgery), injection, by means of a catheter, by means of a suppository, or by means of an implant. An implant can be of a porous, non porous, or gelatinous material, including membranes, such as sialastic membranes, or fibers.

Suppositories generally contain active ingredients in the range of 0.5% to 10% by weight. Oral formulations preferably contain 10% to 95% active ingredient by weight.

A TGF-β response element decoy can be administered before, during, and/or after the administration of one or more therapeutic agents. In one embodiment, a TGF-β response element decoy can first be administered to reduce the expression of a collagen gene, which may decrease the severity of a disease or condition associated with aberrant TGF-β expression and make it more susceptible to another therapeutic agent. In another embodiment, a TGF-β response element decoy can be administered after administration of a therapeutic agent. In yet another embodiment, there can be a period of overlap between the administration of TGF-β response element decoy and/or one or more therapeutic agents.

Demonstration of Therapeutic or Prophylactic Utility. The compositions of the invention are preferably tested in vitro, and then in vivo for the desired therapeutic or prophylactic activity, prior to use in humans. Compositions for use in therapy can be tested in suitable animal model systems prior to testing in humans, including but not limited to rats, mice, chicken, cows, monkeys, rabbits, etc. In vitro assays may be used to demonstrate the therapeutic or prophylactic utility of a composition include, the effect of a composition on a cell line, particularly one characteristic of a specific type of cancer, or a patient tissue sample. The effect of the composition on the cell line and/or tissue sample can be determined utilizing techniques known to those of skill in the art including, but not limited to, rosette formation assays and cell lysis assays. Specifically, liver cancer cell line, breast cancer cell line, such as MDA-MB-231, lymphoma cell line, such as U937, and colon cancer cell line, such as RKO may be used to assess the therapeutic effects of the nucleic acid molecules comprising TGF-β response element. Techniques known to those skilled in the art can be used for measuring cell activities. For example, cellular proliferation can be assayed by 3H-thymidine incorporation assays and trypan blue cell counts.

In yet another specific example, the therapeutic or prophylactic activity of the present therapeutic agent can be assessed by counting the number of apoptotic cells in the treated tissue sample using TUNEL staining method (Hensey C et al., 1998, Program cell death during Xenopus development: a spatio-temporal analysis, Dev Biol 203:36-48; Veenstra, GJ et al., 1998, Non-cell autonomous induction of apoptosis and loss of posterior structures by activation domain-specific interactions of Oct-1 in the Xenopus embryo, Cell Death Differ 5:774-84) and compare with that of control samples.

Test compositions can be tested for their ability to reduce fibrotic disease or tumor formation in patients (i.e., animals) suffering from cancer. Test compositions can also be tested for their ability to alleviate of one or more symptoms associated with fibrotic disease or cancer. Further, test compositions can be tested for their ability to increase the survival period of patients suffering from fibrotic disease or cancer. Techniques known to those of skill in the art can be used to analyze test to function of the test compositions in patients.

In various embodiments, with the invention, in vitro assays which can be used to determine whether administration of a specific composition is indicated, include in vitro cell culture assays in which a patient tissue sample is grown in culture, and exposed to or otherwise administered a composition, and the effect of such composition upon the tissue sample is observed.

The present invention may be better understood by reference to the following non-limiting Examples, which are provided only as exemplary of the invention. The following examples are presented to more fully illustrate the preferred embodiments of the invention. They should in no way be construed, however, as limiting the broader scope of the invention.

### EXAMPLE 1

### Inhibition of In Vitro proα1(I) Collagen Expression

Material and methods. Culturing and treatment of fetal rat lung fibroblasts. Rat fetal lung fibroblasts (RFL-6) were purchased from the American Type Culture Collection (CCL 192; ATCC, Rockville, Md.). RFL-6 fibroblasts were grown in 90% (v/v) Eagle's minimum essential medium (BioWhittaker, Walkersville, Md.) containing 10% (v/v) heat-inactivated fetal bovine serum (FBS) (Hyclone Laboratories, Logan, Utah), 100 U of penicillin per ml, 100 μg of streptomycin per ml, 292 μg of L-glutamine per ml, and 0.22% (w/v) sodium carbonate.

RFL-6 fibroblasts were transiently transfected with ssPTs which are phosphorothioate analogues of DNA having a sulfur in place of an oxygen as one of the non-bridging ligands bound to phosphorous. The phosphorothioate oligodeoxynucleotides contained the TGF-β response element sequence (5′-TGCCCACGGCCAG-3′; SEQ ID NO:1). The complete sequence of the phosphorothioate oligodeoxynucleotides was 5′-AGCCTAACTGCCCACGGCCAGCGACGT-3′ (SEQ ID NO:2). Using the calcium phosphate co-precipitation method (Chen et al., 1987, High-efficiency transformation of mammalian cells by plasmid DNA, Mol. Cell. Biol. 7:2745-2752), cells were transiently transfected with the synthetic oligodeoxynucleotides. The cells were incubated at 37° C. overnight, washed twice with phosphate-buffered saline and placed in AIM V medium. Control cells were treated in the same manner as treated cells except for the absence of oligodeoxynucleotides in the transfection reaction. The presence of transiently transfected oligodeoxynucleotides in nuclei was verified using fluorescent microscopy; ssPTs were synthesized with a 5′-fluorescein label (data not shown).

DNA mobility shift assay. RFL-6 fibroblasts were grown to late log phase. Nuclear protein extracts were prepared by the method of Andrews and Faller (Andrews et al., 1991, A rapid micropreparation technique for extraction of DNA-binding proteins from limiting numbers of mammalian cells, Nucleic Acids Res. 19:2499), and protein concentrations were determined by the method of Lowry et al. (1951, Protein measurement with the folin phenol reagent, J. Biol. Chem. 193:265-275). Single-stranded oligodeoxynucleotides containing the TGF-β element sequence (5′-TGCCCACGGCCAG-3′; SEQ ID NO:1) were synthesized (Integrated DNA Technologies, Coralville, Iowa). A total of 20 μg of the single-stranded oligodeoxynucleotides were annealed with 20 μg of their complementary strands in 200 mM NaCl by heating to 95° for 7 min and then slowly cooled to 4° C. The double-stranded oligodeoxynucleotides were stored at −20° C. The oligodeoxynucleotides were labeled with 32P using the 5′ DNA Terminus Labeling System (Gibco-BRL). Gel shift binding reactions (20 μl) contained 32P-end labeled double-stranded oligodeoxynucleotides (approximately 2.0-3.0×10⁶ cpm/pmol), 10 μg of nuclear protein extract, 1.5 μg of poly (dI-dC) (Pharmacia, Piscataway, N.J.), 90 mM KCl, 1 mM EDTA, 1 mM DTT and 5% glycerol. Reaction mixtures containing 100-fold excess of either cold sPTs or dsPTs were incubated for 30 min at room temperature with nuclear extract and separated on a 6% polyacrylamide gels (19:1 acrylamide to bisacrylamide) in low-ionic-strength buffer (22.25 mM Tris borate, 22.25 mM boric acid, 500 μM EDTA, pH 8.0) for 4 h at 100 V at 4° C. The gels were air-dried and autoradiographed.

Granuloma fibroblast preparation and culture. CBA/J (H-2^{k}) strain female mice were infected with 30 cercariae of the Puerto Rican strain of the worm Schistosoma-mansoni. At 8 weeks of the infection mice were sacrificed and their livers were removed aseptically. Liver granulomas were obtained by gentle homogenization of the tissue. Intact granulomas were washed with RPMI-1640 medium and dispersed by 0.2% type IV collagenase (Sigma, St. Louis, Mo.) and mechanical disruption (Ragheb et al., 1989, Characterization of granuloma T lymphocyte function from Schistosoma mansoni-infected mice, J. Immunol. 142:3239-3246). Dispersed cells were washed three times with Hank's balanced salt (HBSS) solution, were suspended in DMEM with 15% FCS and plated at a concentration of 3.0×10⁷/ml cells onto 100×15 mm plastic tissue culture plates. The plates were incubated for 90 min during which time most of the granuloma macrophages and fibroblasts adhered. The non-adherent cells were removed, the plates were rinsed twice with HBSS and the cells were then incubated in DMEM with 15% FCS for 24 h at 35° C. in 3.7% CO2. A total of 24 h later the floating cells were removed, the plates were rinsed once with HBSS and were further incubated in 10 ml/plate of DMEM and FCS medium. On the 3rd day of incubation, the supernatants containing floating cells were removed, discarded, and fresh media was added. By the 7th day of the culture 50-60% of the cultures contained spindle shaped fibroblasts with some already showing the stellate cell morphology. By day 10 of culture, the macrophages disappeared and the plates were densely covered by cells. About 30% of the cells were enlarged and stained positive with a (α-smooth actin antibody indicating their identity as myofibroblasts.

Collagen synthesis. Seven day old primary schistosomiasis fibroblast cultures were supplemented with 16 μM/plate final concentration of ascorbic acid and the cells were incubated for 48 h. On day 9 of culture the cells were transfected with 50 μg/plate of ssPTs or dsPTs containing the TGF-β response element. Transfection was done dropwise with Ca⁺⁺ buffer as described (Boros, 1989, Immunology of Schistosoma mansoni infection, Clin. Microbiol. Rev. 2:250-269). The cells were incubated for 18 h at 35° C. in 3.7% CO². After the nucleotide was washed away, the plates were rinsed twice with phosphate-buffered saline (PBS) (pH 7.4) and then were replenished with synthetic Aim V medium without serum and incubated for 24 h. The plates were again supplemented with ascorbic acid and with 20 μCi/ml of 5-[³H] proline. After 1.5 h of incubation, the supernatant media was discarded and the cells were removed from the plates by gentle scraping. The cells were washed four times with PBS (pH 7.4), deposited by centrifugation and were stored at −70° C. until further use. For collagen synthesis assays four to six plates were set up for each experimental variable.

Collagenase assay. Frozen sedimented cells were suspended in 2.0 ml of 0.6 M NaCl and 0.05 M Tris-HCl (pH 7.5) in plastic tubes. The cells were sonicated using a Braun-Sonic U sonicator for 2 min on ice. Samples were then placed into boiling water bath for 30 min and the samples were again sonicated for 1 min on ice to disperse clumps. A volume of 500 μl of the homogenate was centrifuged at 8000×g for 20 min in a Sorvall RC-5B centrifuge. The supernatant fluid was used for determining ³H proline incorporation into collagen and non-collagen protein using the protease-free bacterial collagenase assay as described (Newman and Cutroneo, 1978, Glucocorticoids selectively decrease the synthesis of hydroxylated collagen peptides, Mol. Pharmacol. 14:185-198).

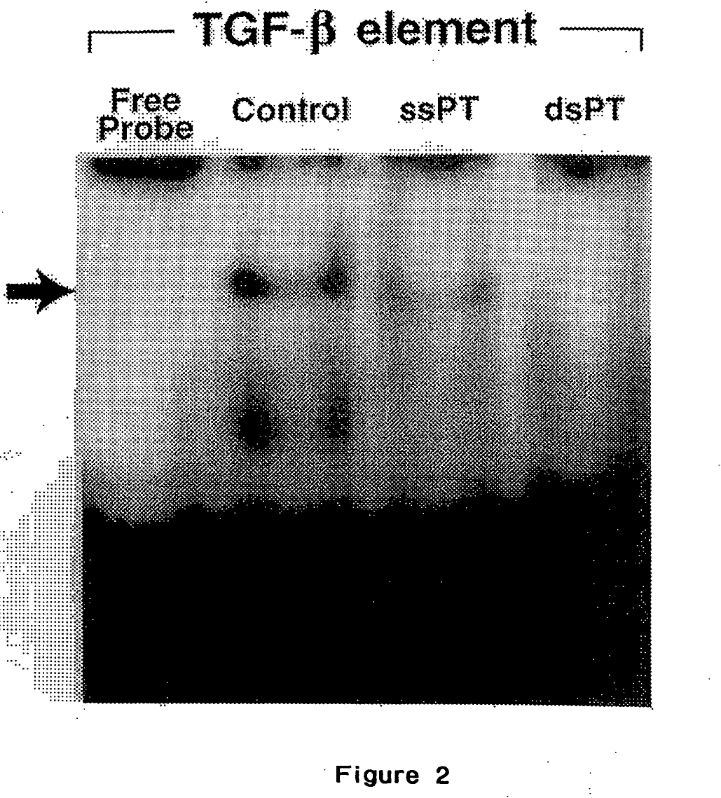

Results. The ability of TGF-β response element decoys to bind TGF-β activator protein was tested by competitive cold oligodeoxynucleotide gel mobility shift analysis. The sense oligonucleotide containing the TGF-β response element, 5′-AGCCTAACTGCCCACGGCCAGCGACGT-3′ (SEQ ID NO: 2), was annealed to its complementary strand and labeled with 32p. The labeled double-stranded oligo was then incubated with control nuclear extract prepared from normal fetal rat lung fibroblasts alone or the nuclear extract was preincubated with either ssPTs or dsPTs. The competing ssPTs diminished the binding of TGF-β activator protein, whereas the dsPTs totally obliterated this binding (FIG. 2).

Because liver fibrosis is a primary risk factor for HCC as well as schistosomiasis, the effect of ssPTs and dsPTs at 50 μg/plate on collagen and noncollagen protein synthesis in primary fibroblasts isolated from hepatic schistosomiasis-induced granulomas were determined (Table 1). The ssPT and dsPT oligos significantly decreased collagen synthesis while slightly increasing the amount of non-collagen protein synthesis which would argue against them being toxic. The combined data are expressed as percent of synthesis of controls since two separate experiments were combined. The original data used to calculate the data in Table 1 were expressed as percent proline incorporation into collagen and non-collagen protein based on proline incorporation into total 8000×g cellular protein to account for possible alterations of the precursor proline pool specific activity.

Table I

The effect of sense single-stranded and double-stranded phosphorothioate oligodeoxynucleotides containing the TGF-β response element on collagen and non-collagen synthesis in fibroblasts isolated from liver granulomas of schistosome-infected mice^{a}

TABLE I
The effect of sense single-stranded and double-stranded
phosphorothioate oligodeoxynucleotides containing the TGF-β
response element on collagen and non-collagen synthesis in
fibroblasts isolated from liver granulornas of
schistosome-infected mice^{a}

Percent synthesis of controls

| Groups | Collagen | Non-collagen |
|---|---|---|
| ssPT (50 μg/plate) | 40.0 ± 16.3 | 115.3 ± 4.5 |
| dsPT (50 μg/plate) | 0.6 ± 0.3* | 109.2 ± 0.3 |
| ^{a}Fibroblasts isolated from dispersed granulomas were grown in serum containing media to late log phase. The cells were then washed twice with PBS and cultured in AIM V synthetic serum-free media. Control cells and ssPT and dsPT transfected cells were incubated in | | |
| # AIM V media for 18 h and washed twice with PBS. Following incubation for 24 h and incubation for 1.5 h before cell collection as described in the text, the cells were collected by scraping, homogenized and the homogenates were heat inactivated, centrifuged at 8000 X g for 20 min and the supernatants were submitted to protease-free bacterial collagenase digestion to determine the percent of proline incorporated into collagen and non-collagen protein. | | |

The values represent the mean ± SEM of four to six cultures in each group.
*Significantly different at P ≦ 0.05 using the Student's t-test.

^{a}Fibroblasts isolated from dispersed granulomas were grown in serum containing media to late log phase. The cells were then washed twice with PBS and cultured in AIM V synthetic serum-free media. Control cells and ssPT and dsPT transfected cells were incubated in AIM V media for 18 h and washed twice with PBS. Following incubation for 24 h and incubation for 1.5 h before cell collection as described in the text, the cells were collected by scraping, homogenized and the homogenates were heat inactivated, centrifuged at 8000×g for 20 min and the supernatants were submitted to protease-free bacterial collagenase digestion to determine the percent of proline incorporated into collagen and non-collagen protein. The values represent the mean ±SEM of four to six cultures in each group. *Significantly different at P≦0.05 using the Student's t-test.

Discussion. ssPTs and dsPTs were effective in inhibiting collagen synthesis in primary fibroblasts isolated from schistosomiasis-induced hepatic granulomas. Because of the central role of collagen synthesis in the wound healing process, the pharmacological control of collagen synthesis is of paramount importance as a possible way to control aberrant wound healing and prevent fibrosis.

Preincubation of control fibroblast nuclear extract with the cold ssPTs diminished the binding of TGF-β activator protein to the TGF-β response element in the 32P double-stranded oligo, while cold dsPTs totally obliterated this binding. Transfection of granuloma fibroblasts with ssPT effectively and significantly diminished collagen synthesis while dsPTs almost totally obliterated collagen synthesis. Neither ssPTs or dsPTs inhibited non-collagen gene expression. A primary risk factor in the onset and progression of HCC is liver fibrosis associated with schistosomiasis infection. In this infection the host's granulomatous inflammatory response to the parasite eggs leads to scar formation, which is the major cause for the pathology of this disease (Wyler, 1991, Schistosomes, fibroblasts, and growth factors: how a worm causes liver scarring, New Biol. 3:734-740). In patients with liver fibrosis complicated with HCC, liver resection and liver transplantation offer radical approaches (Nagasue et al., 1989, Liver resection for hepatocellular carcinoma: results from 150 consecutive patients, Canc. Chem. Pharmacol. 23:S78-S82; Macintosh et al., 1992, Hepatic resection in patients with cirrhosis and hepatocellular carcinoma, Surg. Gyn. Obst. 174:245-254; Mor et al., 1998, Treatment of hepatocellular carcinoma associated with cirrhosis in the era of liver transplantation, Ann. Int. Med. 129:643-653; Moser et al., 2000, Research toward safer resection of the cirrhotic liver, HPB Surg. 11:285-297). The present study indicates that hepatic collagen synthesis and fibrogenesis associated with schistosomiasis liver infection may be inhibited pharmacologically by the use of ssPTs and dsPTs containing the TGF-β regulatory element.

### EXAMPLE 2

### Inhibition of In Vivo Collagen Gene Expression

Materials and methods. Upon arrival the Sprague Dawley rats (Charles River, Wilmington, Mass.) weighed approximately 175 gm prior to a 6-day acclimation period. To implant sterile sponges, an incision was made on the back of each animal above the tail and a trocar was inserted subcutaneously and guided to the dorsothoracic region behind the neck. One cylindrical foam sponge (1.5 cm in diameter×3.5 cm in length pre-soaked in saline; VWR-S/P, Boston, Mass.) was implanted, the trocar was removed and the incision was closed with wound staples.

The animal's care was in accordance with the institutional guidelines set by the Institutional Animal Care Committee at the University of Vermont.

SsPT (0.1 mg), mssPT (0.1 mg) (Research Genetics, Huntsville, Ala.) or dexamethasone (DEX) (0.1 mg) (Steraloids, Wilton, N.H.) were administered 24 hours after sponge implantation directly into the foam sponges. The wild type 27 mer ssPT contained the base sequence 5′-AGCCTAACTGCCCACGGCCAGCGACGT 3′ (SEQ ID NO:2) with the underline portion the TGF-β response element. The TGF-β response element coincides with positions −1636 to −1610 of the rat proα1(I) collagen promoter. The mutated TGF-β response element in the mssPT had the base sequence 5′-TGTGCGCGGCCCT 3′ (SEQ ID NO:3) where the underlined bases represent the changed bases in the TGF-β response element. An initial intrasponge injection was made 24 hours after sponge implantation and was followed by a second injection 24 hours later. The oligodeoxynucleotide presumably gets from the sponge matrix into cells forming the capsule through passive transport. Twenty-four hours after the second injection of drug, the animals were euthanized by an overdose of pentobarbital. Therefore, animals, except for the acute experiment, were injected for 2 daily injections starting 24 hours after sponge implantation on day 2 and 3. On day 4 animals were euthanized. For the acute treatment, 4-day-old granulomas were treated with 2 injections at 3 hours intervals and euthanized after administering ³H-proline 1 hour prior to killing. The granulomatous tissue encapsulating the sponge was remove and weighed. The granuloma weights and the granuloma:body weight ratios were determined as index's of antifibrotic activity.

Collagen and noncollagen protein synthesis. In experiments to assess collagen and noncollagen protein synthesis, 1 hour prior to killing, each animal was given an intrasponge injection of 100 μCi of 5-3H-proline (spec. act. 29 Ci/mmol; Amersham, Boston, Mass.). The sponges encapsulated with granulomatous tissue were dissected from the surrounding tissue and the granulomatous tissue was separated from the sponge. The granulomatous tissue was homogenized in 0.6 M NaCl containing 0.05 M Tris HCI (pH 7.1) using a Polytron homogenizer. The samples were heat inactivated in a boiling water bath, rehomogenized to disperse clumps and centrifuged at 8000×g for 30 minutes at 4° C. The supernatants were then submitted to protease-free bacterial collagenase digestion as previously described (Guzman et al., 1976, Collagen lysyl hydroxylation occurs in the cisternae of the rough endoplasmic reticulum. Arch Biochem Biophys 172:449-54). Following incubation, the samples were precipitated with 2 ml of 10% (w/v) trichloroacetic acid and the sample was filtered through a glass fiber filter and washed 3× with 5% (w/v) trichloroacetic acid. The percent of 3H-proline incorporated into collagen (i.e., collagenase-digestible CPM) was calculated based on total 3H-proline incorporation and the percent of ³H-proline incorporated into noncollagen protein (i.e., the CPM remaining after collagenase digestion) was calculated based on total ³H-proline incorporation. The collagenase digestible protein was determined by subtracting the collagenase digestible protein from the total ³H-proline incorporated.

Statistical analysis. Statistical evaluation of data was determined by linear regression analysis or the Student's t-test. Significant differences require p≦0.05.

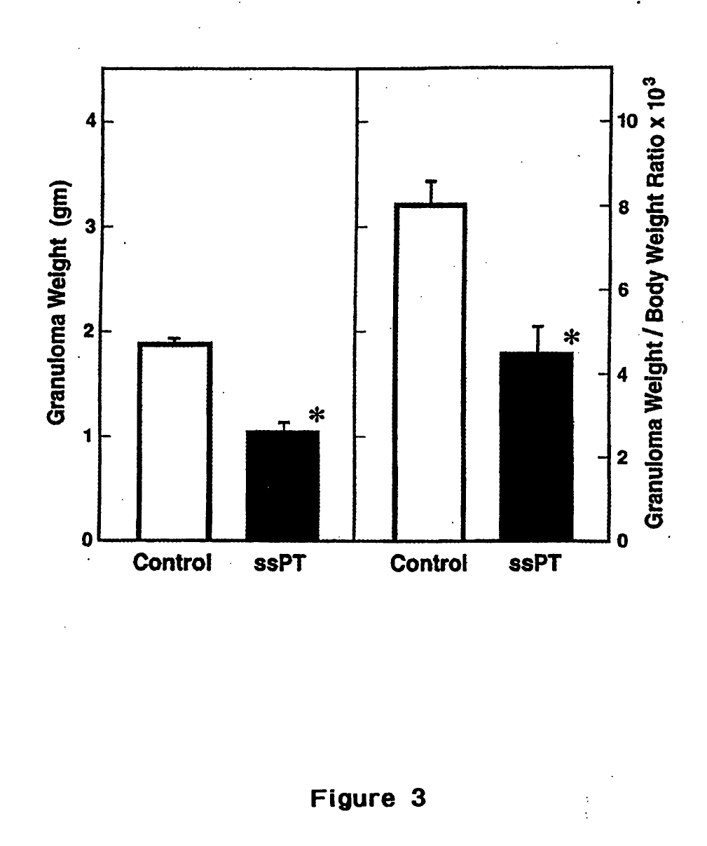

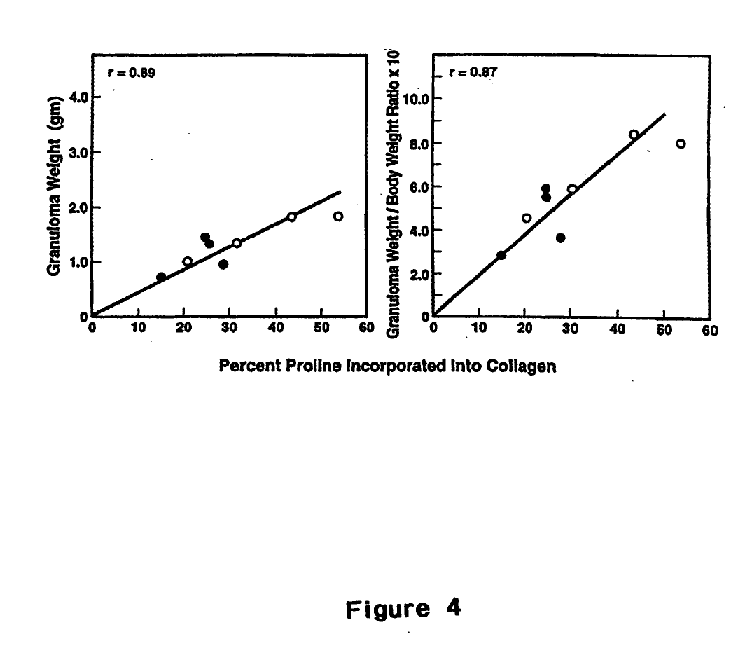

Results. The weight gain of the animals was not significantly different between the control (22.6±3.7 gm, mean ±SE) and the ssPT treated group (18.0±2.1 gm), by the Student's t-test. However, the granuloma weights and granuloma:body weight ratios were both statistically significantly decreased in the ssPT treated group as compared to control (FIG. 3). The results of this experiment described in FIG. 3 were carefully evaluated because the granuloma weights were normalized based on the animals body weight to account for possible growth changes among individual animals. These two parameters were strongly and linearly correlated with an r-value equal to 0.98. Furthermore, the decrease of granuloma growth was closely correlated to the inhibition of collagen synthesis as seen in the experiment described in FIG. 4. The percent of ³H-proline incorporated into collagen was linearly correlated to granuloma growth (FIG. 4).

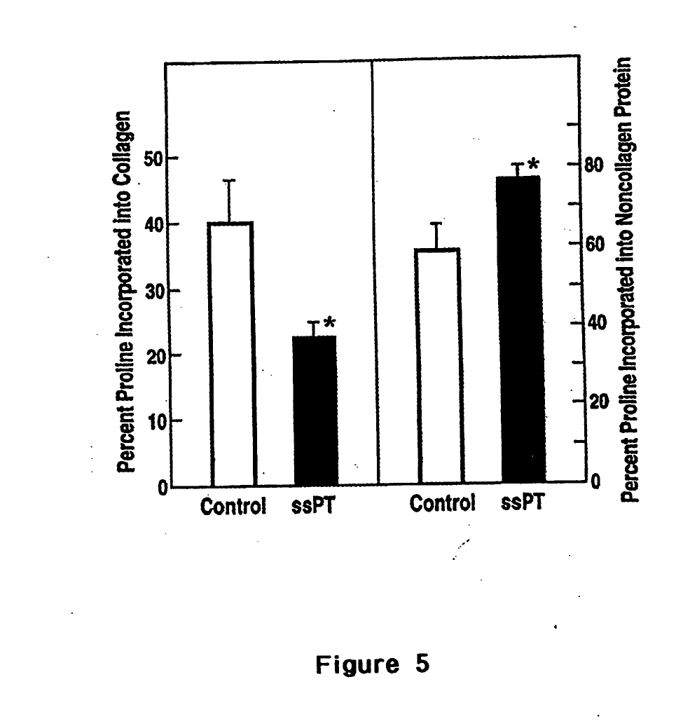

While collagen synthesis was significantly decreased by ssPT treatment, noncollagen protein synthesis was significantly increased (FIG. 5). The percent of collagen synthesis of the control group was 40.2±6.2 (mean ±S.E.) of total proline incorporation while that of the ssPT treated group was 22.2±2.4. The value for noncollagen protein synthesis for the control group was 59.8±6.2 (mean ×S.E.) while that of the ssPT treated group was 77.8±2.4. MssPT given at the same dose did not affect granuloma growth, collagen or noncollagen protein synthesis. DEX given for 2 daily injections at 0.1 mg/sponge did not affect either collagen or noncollagen protein synthesis.

Discussion Appropriately constructed ssPTs are able to regulate the fibrotic response to sponge implantation in vivo. This demonstrates that the TGF-β response element which is located in the 5′-flanking region of the proα1(I) collagen gene is a key determinant of collagen gene expression and collagen synthesis in vivo. TGF-β is involved in the fibrotic response through regulating in vivo collagen synthesis at the molecular level. The effect of ssPT on granuloma growth and collagen synthesis is contrasted to the inability of mssPT and DEX to affect the same parameters. Furthermore, ssPT treatment does not nonspecifically decrease protein synthesis because ssPTs treatment of granulation tissue fibroblasts did not affect the basal levels of either collagen or noncollagen protein sythesis. In addition, in the present study ssPT in vivo increased noncollagen protein synthesis while these oligodeoxynucleotides decreased collagen synthesis and granuloma/ body weight ratio. This latter parameter of granuloma growth normalizes for any variation of body weight between animals.

### EXAMPLE 3

### Inhibition of Collagen Synthesis by Double Stranded (dsPT) Oligodeoxynucleotides Containing the TGF-β Element—Antifibrotic Intervention

Ten day old granuloma hepatic stellate cells that secrete TGF-β were transfected with 50 μg/10 ml double stranded (dsPT) oligodeoxynucleotides containing the TGF-β element. Untreated monolayers produced 15-20 percent collagen in 48 hr. The dsPT decoy-treated cultures totally suppressed collagen but not noncollagen protein production.

### EXAMPLE 4

### In Vivo—Antifibrotic Intervention.

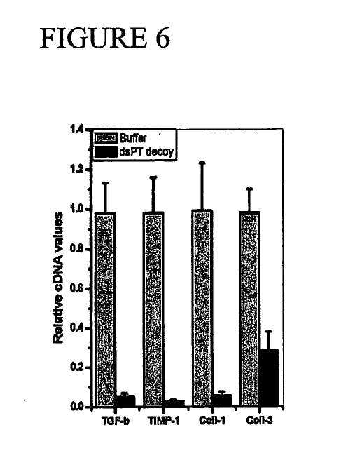

Mice at 6.2 weeks of infection received intrahepatically 800 μg in 0.1 ml of dsPT followed by 3 daily iv injections of 200 μg in 0.1 ml. Control mice received buffer. Twenty four hours after the last injection mice were sacrificed and their livers were examined for the expression of TGF-β. TIMP-1 and collagen I and collagen III messages, as well as total hydroxyproline content. Referring to FIG. 6 the 5 recipients of the dsPT showed a virtual elimination of TGFβ, TIMP-1 and collagen I messages, and collagen III message was significantly reduced. This indicates a coordinate regulation by TGF-β element of two collagen isotypes. Downregulation of TIMP-1 expression further enhances the anti-fibrotic effect. Total collagen as measured by hydroxyproline measurement diminished by 20% compared with buffer control. Significantly, dsPT decoy treatment also totally suppressed intragranulomatous collagen synthesis as measured by ³H labeled praline uptake by intact granulomas. In the control samples, 10% of the collagen was synthesized.

All references cited herein are specifically incorporated by reference as if fully set forth herein.

Having hereinabove disclosed exemplary embodiments of the present invention, those skilled in the art will recognize that this disclosure is only exemplary such that various alternatives, adaptations, and modifications are within the scope of the invention, and are contemplated by the Applicants. Accordingly, the present invention is not limited to the specific embodiments as illustrated above, but is defined by the following claims.

1-9. (canceled)

10. A method of regulating expression of a gene comprising a TGF-β response element comprising administering to a cell a nucleic acid molecule comprising SEQ ID NO: 1.

11. The method of claim 10, wherein the nucleic acid molecule is capable of binding to a transcription factor that is modulated by TGF-β.

12. The method of claim 10, wherein the nucleic acid molecule is a double stranded phosphorothioate nucleic acid molecule.

13. The method of claim 10, wherein the double-stranded nucleic acid molecule comprises two or more TGF-β response elements.

14. A method of regulating expression of a gene comprising a TGF-β response element comprising administering to a cell a nucleic acid molecule comprising SEQ ID NO: 2.

15. The method of claim 14, wherein the nucleic acid molecule is a double stranded phosphorothioate nucleic acid molecule.

16. The method of claim 14, wherein the double-stranded nucleic acid molecule comprises two or more TGF-β response elements.

17. A method of regulating expression of a gene comprising a TGF-β response element comprising administering to a cell a derivative of a nucleic acid molecule comprising SEQ ID NO: 1.

18. A method of regulating expression of a gene comprising a TGF-β response element comprising administering to a cell a derivative of a nucleic acid molecule comprising SEQ ID NO: 2.

19. A method of treating or preventing liver cirrhosis or tissue fibrosis in a human comprising administering to said human a nucleic acid molecule comprising SEQ ID NO: 1.

20. The method of claim 19, wherein the nucleic acid molecule is a double stranded phosphorothioate nucleic acid molecule.

21. The method of claim 19, wherein the nucleic acid molecule comprises two or more TGF-β response elements.

22. The method of claim 19, wherein said administration is by intravenous infusion.

23. The method of claim 19, wherein the nucleic acid molecule is administered for a period of 2 to 13 days.

24. The method of claim 19, wherein the nucleic acid molecule is administered for a period of 14 to 28 days.

25. The method of claim 19, further comprising administering about 0.01 to about 10 mg/kg/day of the nucleic acid molecule.

26. The method of claim 19, further comprising administering about 10 to about 50 mg/kg/day of the nucleic acid molecule.

27. A method of treating or preventing liver cirrhosis or tissue fibrosis in a human comprising administering to said human, in which such treatment or prevention is desired, a nucleic acid molecule comprising SEQ ID NO: 2.

28. The method of claim 27, wherein the nucleic acid molecule is a double stranded phosphorothioate nucleic acid molecule.

29. The method of claim 27, wherein the nucleic acid molecule comprises two or more TGF-β response elements.

30. The method of claim 27, wherein said administration is intravenous infusion.

31. The method of claim 27, wherein the nucleic acid molecule is administered for a period of 2 to 13 days.

32. The method of claim 27, wherein the nucleic acid molecule is administered for a period of 14 to 28 days.

33. The method of claim 27, further comprising administering about 0.01 to about 10 mg/kg/day of the nucleic acid molecule.

34. The method of claim 27, further comprising administering about 10 to about 50 mg/kg/day of the nucleic acid molecule.

35-43. (canceled)


SEQUENCE LISTING (3 sequences)

SEQ ID NO: 1 - DNA, 13 bp, Artificial Sequence
  Description of Artificial Sequence Synthetic oligonucleotide
tgcccacggccag

SEQ ID NO: 2 - DNA, 27 bp, Artificial Sequence
  Description of Artificial Sequence Synthetic oligonucleotide
agcctaactgcccacggccagcgacgt

SEQ ID NO: 3 - DNA, 13 bp, Artificial Sequence
  Description of Artificial Sequence Synthetic oligonucleotide
tgtgcgcggccct